(19) United States
(12) Patent Application Publication
(10) Pub. No.: US 2010/0047216 A1
(43) Pub. Date: Feb. 25, 2010

(54) COMPOSITIONS OF LATE PASSAGE MESENCHYMAL STEM CELLS (MSCS)

(76) Inventors: Glenn Gaudette, Holden, MA (US); Irina A. Potapova, East Setauket, NY (US); Peter R. Brink, Setauket, NY (US); Ira S. Cohen, Stony Brook, NY (US); Richard B. Robinson, Cresskill, NJ (US); Michael R. Rosen, New York, NY (US)

Correspondence Address: KENYON & KENYON LLP, ONE BROADWAY, NEW YORK, NY, 10004, US

(21) Appl. No.: 12/374,027

(22) PCT Filed: Jul. 20, 2007
(86) PCT No.: PCT/US07/16429

§ 371 Date: Nov. 3, 2009

Related U.S. Application Data
(60) Provisional application No. 60/832,516, filed on Jul. 21, 2006.

(51) Int. Cl. A61K 35/12; C12N 5/0775; C12N 5/10; C12N 5/00

(52) U.S. Cl. 424/93.21; 435/366; 435/346

(57) ABSTRACT

The present invention provides methods and compositions relating to the use of late passage mesenchymal stem cells (MSCs) for treatment of cardiac disorders. Such late passage MSCs may be administered to the myocardium of a subject for induction of native cardiomyoctye proliferation and repair of cardiac tissue. Additionally, the late passage MSCs may be genetically engineered to express a gene encoding a physiologically active protein of interest and/or may be incorporated with small molecules for delivery to adjacent target cells through gap junctions. The late passage MSCs of the invention may be used to provide biological pacemaker activity and/or provide a bypass bridge in the heart of a subject afflicted with a cardiac rhythm disorder. The biological pacemaker activity and/or bypass bridge may be provided to the subject either alone or in tandem with an electronic pacemaker.

This research was supported by USPHS-NHLBI grants HL-28958 and HL-67101. The United States Government may have rights in this invention.

### INTRODUCTION

The present invention provides methods and compositions relating to the use of late passage mesenchymal stem cells (MSCs) for treatment of cardiac disorders. Such late passage MSCs may be administered to the myocardium of a subject for induction of native cardiomyoctye proliferation and repair of cardiac tissue. Additionally, the late passage MSCs may be genetically engineered to express a gene encoding a physiologically active protein of interest and/or may be incorporated with small molecules for delivery to adjacent target cells through gap junctions. The late passage MSCs of the invention may be used to provide biological pacemaker activity and/or provide a bypass bridge in the heart of a subject afflicted with a cardiac rhythm disorder. The biological pacemaker activity and/or bypass bridge may be provided to the subject either alone or in tandem with an electronic pacemaker. The invention is based on the discovery that late passage MSCs have lost their ability to differentiate into cells of osteogenic, chondrogenic or adipogenic lineages, thereby enhancing their safety and efficacy.

### BACKGROUND OF INVENTION

Heart failure is a notoriously progressive disease, despite medical management. The increasing gap between the incidence of end-stage heart failure and surgical treatment is due, in great part, to the shortage of donor organs. Thus, there is a need for alternative approaches for treatment of damaged heart tissue that is not dependent of the availability of donor organs.

Although mesenchymal stem cells can be used as a vehicle for gene delivery to the cardiac syncytium, one significant drawback to the use of such cells is their ability to differentiate into different cell types of osteogenic, chondrogenic or adipogenic lineages. The present invention is based on the discovery that late passage MSCs have lost their ability to differentiate along different lineages thus increasing safety and efficacy. Accordingly, the present invention provides novel methods and compositions for treatment of cardiac disorders based on the use of late passage MSCs.

### SUMMARY OF THE INVENTION

The present invention provides methods and compositions relating to the use of late passage MSCs for treatment of cardiac disorders. The invention is based on the discovery that late passage MSCs have lost their ability to differentiate into cells of osteogenic, chondrogenic or adipogenic lineages, thereby enhancing their safety and efficacy.

Accordingly, the present invention relates to compositions comprising late passage MSCs that are substantially incapable of differentiation. In a preferred embodiment, the late passage MSCs have been passaged at least nine times. Additionally, the late passage MSCs of the invention express CD29, CD44, CD54 and HLA class I surface markers while failing to express CD14, CD45, CD34 and HLA class II surface markers.

Compositions comprising late passage MSCs may be used for regenerating myocardium through stimulation of native cardiomyocyte proliferation. Specifically, the invention relates to the use of late passage MSCs to promote an increase in the number of cells in the myocardium through increased proliferation of native cardiac progenitor cells resident in the myocardium; stimulation of myocyte proliferation; and stimulation of differentiation of host cardiac progenitor stem cells into cardiac cells, for example. Such an increase in cell number results predominantly from stimulation of the native myocardium cells by factors produced by the administered late passage MSCs. In another embodiment of the invention, scaffolds designed for implantation may be engineered to contain exogenously added late passage MSCs, which are capable of stimulating cardiomyocyte proliferation.

In an embodiment of the invention, late passage MSCs may comprise an exogenous molecule including, but are not limited to, oligonucleotides, polypeptides, or small molecules, and wherein said late passage MSC is capable of delivering said exogenous molecule to an adjacent cell. Delivery of the exogenous molecule to adjacent cells may be used to stimulate cardiomyocyte proliferation, cardiac repair or provide biological pacemaker activity.

The present invention provides a method of delivering an oligonucleotide, protein or small molecule into a target cell comprising: (i) introducing the oligonucleotide, protein, or small molecule into a late passage MSC and (ii) contacting the target cell with the late passage MSC under conditions permitting the late passage MSC to form a gap junction channel with the target cell, whereby the oligonucleotide, protein, or small molecule is delivered into the target cell from the late passage MSC.

In yet another embodiment of the invention, late passage MSCs may be genetically engineered to express a protein or oligonucleotide of interest. Such proteins or oligonucleotides may be those capable of stimulating cardiomyocyte proliferation, cardiac repair or providing biological pacemaker activity.

In a specific embodiment of the invention, the late passage MSCs are engineered to functionally expresses a hyperpolarization-activated, cyclic nucleotide-gated (HCN) ion channel, and wherein expression of the HCN channel is effective to induce a pacemaker current in said cell. In an embodiment of the invention, the expressed HCN channel is a mutant or chimeric HCN channel. Chimeric HCN channels are those HCN channels comprising an amino terminal portion, an intramembrane portion, and a carboxy terminal portion, wherein the portions are derived from more than one HCN isoform. In a preferred embodiment of the invention, the chimeric or mutant HCN charnel provides an improved characteristic, as compared to a wild-type HCN channel, selected from the group consisting of faster kinetics, more positive activation, increased levels of expression, increased stability, enhanced cyclic nucleotide responsiveness, and enhanced neurohumoral response. Such late passage MSCs may also be engineered to functionally expresses a MiRP1 beta subunit along with an HCN channel.

In addition, this invention provides a biological pacemaker comprising a late passage MSC which functionally expresses an HCN ion channel or a mutant or chimera thereof, with or without a MiRP1 beta subunit or a mutant thereof, at a level effective to induce a pacemaker activity in the cell when implanted into a subject.

The present invention further relates to a pharmaceutical composition comprising a population of late passage MSCs, substantially incapable of differentiation, and a pharmaceutically acceptable carrier. In a specific embodiment of the invention, the late passage MSCs comprise an exogenous molecule wherein the exogenous molecule is an oligonucleotide, polypeptide, or small molecule, and wherein said late passage MSC is capable of delivering said exogenous molecule to an adjacent cell. In yet another embodiment of the invention, the late passage MSCs are genetically engineered to express an oligonucleotide or a polypeptide.

The present invention further provides a bypass bridge comprising gap junction-coupled late passage MSCs, which are substantially incapable of differentiation, the bridge having a first end and a second end, both ends capable of being attached to two selected sites in a heart, so as to allow the propagation of an electrical signal across the tract between the two sites in the heart. In a specific embodiment of the invention, the first end is capable of being attached to the atrium and the second end capable of being attached to the ventricle, so as to allow propagation of a pacemaker and/or electrical current/signal from the atrium to travel across the tract to excite the ventricle.

In yet another embodiment of the invention, the cells of the bypass tract functionally express at least one protein selected from the group consisting of: a cardiac connexin; an alpha subunit and accessory subunits of a L-type calcium channel; an alpha subunit with or without the accessory subunits of a sodium channel; and a L-type calcium and/or sodium channel in combination with the alpha subunit of a potassium channel, with or without the accessory subunits of the potassium channel.

In another embodiment of the invention, the cells of the bypass bridge functionally expresses: (i) a hyperpolarization-activated, cyclic nucleotide-gated (HCN) ion channel capable of generating a pacemaker current in said cell, (ii) a chimeric HCN channel comprising an amino terminal portion, an intramembrane portion, and a carboxy terminal portion, wherein the portions are derived from more than one HCN isoform, and wherein the expressed chimeric HCN channel generates a pacemaker current in said cell, or (c) a mutant HCN channel wherein the mutant HCN channel generates a pacemaker current in said cell.

Further, the present invention provides the use of the MSCs in a tandem pacemaker system comprising (1) an electronic pacemaker; (2) a biological pacemaker comprising an implantable late passage MSC that functionally expresses (a) an HCN ion channel, or (b) a chimeric HCN channel, or (c) a mutant HCN channel wherein the expressed HCN, chimeric HCN or mutant HCN channel generates an effective pacemaker current when said cell is implanted into a subject's heart; (3) and/or a bypass bridge comprising a strip of gap junction-coupled late passage MSCs having a first end and a second end, both ends capable of being attached to two selected sites in a heart, so as to allow the transmission of a pacemaker and/or electrical signal/current across the tract between the two sites in the heart. In an embodiment of the invention, the biological pacemaker of the tandem system, comprises at least about 5,000 late passage MSCs. In another embodiment of the invention, the biological pacemaker comprises at least about 200,000 late passage MSCs. In another embodiment of the invention, the tandem pacemaker system comprises at least about 700,000 late passage MSCs.

The present invention provides methods for promoting cardiac repair in a subject, comprising administering to said subject an effective amount of late passage MSCs thereby promoting cardiac repair. The methods of the invention may be used to treat a variety of different cardiac disorders, including but not limited to, myocardial dysfunction or infarction, cardiac rhythm disorders, disorders at the sinoatrial node and disorders of the atrioventricular node. In patients in whom biological pacemaker activity and/or a bypass bridge has/have been provided, the subject may also be provided with an electronic pacemaker.

### BRIEF DESCRIPTION OF THE FIGURES

FIG. 1. Fat vacuoles in 4^{th} passage hMSCs exposed to adipogenic differentiation.

FIG. 2. 4^{th} passage hMSCs were first transfected with the PIRES-HCN2 plasmid followed by exposure to adipogenic differentiation. There are fewer cells with fat vacuoles but staining with oil red O still demonstrates a significant number of positive (red) cells.

FIG. 3. Minimal adiopogenic differentiation of 9^{th} passage non-transfected hMSCs is demonstrated by the presence of few fat vacuoles.

FIG. 4. Absence of adipogenic differentiation in 9^{th} passages hMSCs transfected with the PIRES-HCN2 plasmid.

FIG. 5. Western blots demonstrating abundant connexin 43 expression in 3^{rd} and 8^{th} passage hMSCs (right panel) and 3, 5 and 9^{th} passage hMSCs and 2^{nd} passage canine hMSCs (right panel).

FIG. 6. Caspase activation assay for apoptotic cells. Minimal activation is observed for hMSCs at passages 3, 5 or 10 indicating no predisposition to apoptosis.

FIG. 7. DNA analysis by gel electrophoresis of passages 2, 3 and 9 hMSCs. There is no DNA fragmentation, indicating that these passage hMSCs do not have a predisposition to apoptosis.

FIG. 8. Phenotypic characterization of hMSCs of passage 5 and 10 by flow cytometry demonstrating the presence of CD44 and CD54 antigen in both sets of cells.

FIG. 9 Phenotypic characterization of hMSCs of passages 5 and 10 by flow cytometry; HLA class I markers but not HLA class II markers are present on both sets of cells.

FIG. 10. Phenotypic characterization of hMSCs of passage 5 and 10 by flow cytometry; there is CD29 but not CD34 antigen in both sets of cells.

FIG. 11. Phenotypic characterization of hMSCs of passage 5 and 10 by flow cytometry; CD14 and CD45 antigens are absent in both sets of cells.

FIG. 12. Expression of HCN2-induced I_{f} like current is the same in cells from passages 5 and 9 transfected with the PIRES-HCN2 plasmid: FIG. 12A. Fluorescence images of passage 5 cells (upper two panels) and sample current record from patch clamp recordings (lower panel); FIG. 12B. Fluorescence images of passage 9 cells (upper 2 panels) and sample current record from patch clamp recordings (lower panel); FIG. 12C. Histogram comparing the capacitance (left 2 bars) and the HCN2-induced current density (right two bars). There is no significant difference in either parameter between hMSCs from passage 5 and 9.

FIG. 13. Biophysical properties of passage 5 and passage 9 cells expressing HCN2-induced current are very similar. FIG. 13A. Comparison of current records of HCN2-incuded current in passage 5 (left panel) and passage 9 (right panel) hMSCs. The current records are very similar. FIG. 13B. Activation curves obtained from passage 5 (left panel) and passage 9 (right panel) cells show the same midpoint of activation.

FIG. 14. Alignment of mammalian HCN1 polypeptide sequences. The mouse (SEQ ID NO:9), rat (SEQ ID NO:10), human (SEQ ID NO:11), rabbit (SEQ ID NO:12) and guinea pig (partial sequence; SEQ ID NO: 13) HCN1 polypeptide sequences are aligned for maximum correspondence.

FIG. 15. Amino acid sequence of the human HCN212 chimeric channel. The shaded N-terminal portion of the sequence is derived from hHCN2; the underlined intramembranous portion from hHCN1; and the C-terminal portion (without shading or underline) from hHCN2. The amino acid sequence of the hHCN212 chimeric channel is set forth in SEQ ID NO:2. This 889-amino acid long chimeric hHCN212 sequence shows 91.2% identity with the 863-amino acid long mHCN212 sequence in 893 residues overlap when aligned for maximum correspondence.

FIG. 16. Amino acid sequence of the mouse HCN212 chimeric channel. The shaded N-terminal portion of the sequence is derived from mouse HCN2; the underlined intramembranous portion from mouse HCN1; and the C-terminal portion (without shading or underline) from mouse HCN2. The amino acid sequence of the mouse HCN212 chimeric channel is set forth in SEQ ID NO:6. This 863-amino acid long chimeric mHCN212 sequence shows 91.2% identity with the 889-amino acid long hHCN212 sequence in 893 residues overlap when aligned for maximum correspondence.

FIG. 17. Alignment of mammalian HCN2 polypeptide sequences. The mouse (SEQ ID NO:14), rat (SEQ ID NO:15), human (SEQ ID NO:16) and dog (partial sequence; SEQ ID NO:17) HCN2 polypeptide sequences are aligned for maximum correspondence.

FIG. 18. Alignment of mammalian HCN4 polypeptide sequences. The mouse (SEQ ID NO:18), rat (SEQ ID NO:19), human (SEQ ID NO:20), rabbit (SEQ ID NO:21) and dog (partial sequence; SEQ ID NO:22) HCN4 polypeptide sequences are aligned for maximum correspondence.

### DETAILED DESCRIPTION OF THE INVENTION

The present invention provides methods and compositions relating to the use of late passage mesenchymal late passage MSCs (MSCs) for treatment of cardiac disorders. The methods and compositions of the invention may be used in the treatment of cardiac disorders including, but not limited to, arrhythmias, myocardial dysfunction or infarction. As described in detail below, such late passage MSCs may be used to stimulate native cardiomyocyte proliferation. Additionally, late passage MSCs may be incorporated with small molecules, such as small nucleic acid molecules, for delivery to adjacent target cells through gap junctions. Further, late passage MSCs may be genetically engineered to express one or more genes encoding physiologically active proteins of interest. Such proteins include, for example, those proteins capable of stimulating cardiomyocyte proliferation, cardiac repair, proteins capable of providing biological pacemaker activity such as wild type, mutant and chimeric HCN ion channels and the HCN beta subunit MiRPI. In yet another embodiment of the invention late passage MSCs can be used to provide a bypass bridge to those subjects afflicted with sinoatrial or atrioventricular node disorders. The use of biological pacemakers and bypass bridges may be administered to a subject in need of pacemaker function either alone or in tandem with an electronic pacemaker.

### Late Passage MSCs

The present invention relates to methods and compositions relating to the use of late passage MSCs, which are substantially unable to differentiate, for treatment of cardiac disorders. As used herein, “late passage MSCs” are those cells that have been passaged at least nine times. Additionally, the late passage MSCs of the invention express CD29, CD44, CD54 and HLA class I surface markers while failing to express CD14, CD45, CD34 and HLA class II surface markers. In an embodiment of the invention, the late passage MSCs are mammalian in origin. In a preferred embodiment of the invention, the MSCs are derived from a human adult. Substantially unable to differentiate means that virtually all cells in a particular culture will not be able to differentiate. hMSCs that have been passaged at least nine times and that express CD29, CD44, CD54 and HLA class I surface markers while failing to express CD14, CD45, CD34 and HLA class II surface markers are considered “substantially not able to differentiate” as virtually, if not all, cells failed to differentiate into cells of osteogenic, chondrogenic or adipogenic lineages.

Human MSCs (Poietics™ hMSCs) to be used in the practice of the invention can be purchased from any reputable supplier such as Clonetics/Bio Whittaker (Walkersville, Md.). Alternatively, late passage MSCs may be derived from bone marrow aspirates from the subject or from a healthy volunteer. For example, 10 ml of marrow aspirate is collected into a syringe containing 6000 units of heparin to prevent clotting, washed twice in phosphate buffer solution (PBS), added to 20 ml of control medium (DMEM containing 10% FBS), and then centrifuged to pellet the cells and remove the fat. The cell pellet is then resuspended in control medium and fractionated at 1100 g for 30 min on a density gradient generated by centrifugation of a 70% percoll solution at 13000 g for 20 minutes. The mesenchymal stem cell-enriched, low density fraction is collected, rinsed with control medium and plated at a density of 10⁷ nucleated cells per 60 mm² dish. The mesenchymal late passage MSCs are then cultured in control medium at 37° C. in a humidified atmosphere containing 5% CO₂. A preferred culturing medium is a medium that prevents/inhibits differentiation, such as a medium sold by Cambrex Corporation, referred to as MSCGM medium.

Furthermore, antibodies that bind to cell surface markers selectively expressed on the surface of late passage MSCs may be used to identify or enrich for populations of MSCs using a variety of different methods. Such markers include, for example, CD29, CD44 and CD54 which are expressed on the surface of late passage MSCs.

The advantages to using MSCs are that they do not require an endoderm for differentiation, are easy to culture, do not require an expensive cytokine supplement and have minimal immunogenicity. The advantages to using late passage hMSCs is that they have lost their ability to differentiate into osteogenic, chondrogenic or adipogenic lineages thereby enhancing their efficacy and safety.

### Use of Late Passage Mesenchymal Cells for Stimulation of Cardiac Repair

Late passage MSCs are capable of inducing native cardiomyocytes to enter the cell cycle. Accordingly, the present invention encompasses methods for regenerating myocardium in a mammal comprising administering late passage MSCs to the myocardium in a quantity sufficient to induce native cardiomyocytes to enter the cell cycle. Specifically, the invention relates to the use of late passage MSCs to promote an increase in the number of cells in the myocardium through increased proliferation of native cardiac progenitor cells resident in the myocardium; stimulation of myocyte proliferation; and/or stimulation of differentiation of host cardiac progenitor late passage MSCs into cardiac cells, for example. Such an increase in cell number results predominantly from stimulation of the native myocardium cells by factors produced by the administered late passage MSCs.

Prior to administration of the late passage MSCs, the cells may be genetically engineered using techniques well known in the art to express proteins that further enhance the ability of such cells to enhance cardiomyocyte proliferation. In a non-limiting embodiment of the invention, the late passage MSCs are engineered to express the Wnt-5A protein, which enhances cardiomyocyte proliferation. Such techniques include, for example, in vitro recombinant DNA techniques, synthetic techniques, and in vivo genetic recombination. (See, for example, the techniques described in Sambrook J et al. 2000. Molecular Cloning: A Laboratory Manual (Third Edition), and Ausubel et al (1996) Current Protocols in Molecular Biology John Wiley and Sons Inc., USA). Any of the methods available in the art for gene delivery into a host cell can be used according to the present invention to deliver genes into the late passage MSCs. Such methods include electroporation, lipofection, calcium phosphate mediated transfection, or viral infection. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215.

The present invention further provides pharmaceutical compositions comprising late passage MSCs and a pharmaceutically acceptable carrier. Pharmaceutically acceptable carriers are well known to those skilled in the art and include, but are not limited to, 0.01-0.1M and preferably 0.05M phosphate buffer, phosphate-buffered saline (PBS), or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Preservatives and other additives, such as, for example, antimicrobials, antioxidants and chelating agents may also be included with all the above carriers.

Late passage MSCs can also be incorporated or embedded within scaffolds that are recipient-compatible and which degrade into products that are not harmful to the recipient. These scaffolds provide support and protection for late passage MSCs that are to be transplanted into the recipient subjects. Natural and/or synthetic biodegradable scaffolds are examples of such scaffolds. Accordingly, the present invention provides methods for promoting cardiac repair, wherein late passage MSCs are incorporated within scaffolds, prior to transplantation into a subject in need of cardiac repair.

A variety of different scaffolds may be used successfully in the practice of the invention. Such scaffolds are typically administered to the subject in need of treatment as a transplanted patch. Preferred scaffolds include, but are not limited to biological, degradable scaffolds. Natural biodegradable scaffolds include collagen, fibronectin, and laminin scaffolds. Suitable synthetic material for a cell transplantation scaffold must be biocompatible to preclude migration and immunological complications, and should be able to support extensive cell growth and differentiated cell function. It may also be resorbable, allowing for a completely natural tissue replacement. The scaffold should be configurable into a variety of shapes and should have sufficient strength to prevent it from collapsing or from pressure-induced bursting upon implantation. Recent studies indicate that the biodegradable polyester polymers made of polyglycolic acid fulfill all of these criteria, as described by Vacanti, et al. J. Ped. Surg. 23:3-9 (1988); Cima, et al. Biotechnol. Bioeng. 38:145 (1991); Vacanti, et al. Plast. Reconstr. Surg. 88:753-9 (1991). Other synthetic biodegradable support scaffolds include synthetic polymers such as polyanhydrides, polyorthoesters, and polylactic acid.

In an embodiment of the invention, the scaffold is derived from porcine urinary bladder. Alternatively, in a preferred embodiment of the invention the scaffold is derived from bovine pericardium. In a specific embodiment of the invention, Veritas®, which is derived from bovine pericardium, may be utilized.

Attachment of the cells to the scaffold polymer may be enhanced by coating the polymers with compounds such as basement membrane components, agar, agarose, gelatin, gum arabic, collagens types I, II, III, IV and V, fibronectin, laminin, glycosaminoglycans, mixtures thereof, and other materials known to those skilled in the art of cell culture. Additionally, such scaffolds may be supplemented with additional components capable of stimulating cardiomyocyte proliferation. Additionally, angiogenic and other bioactive compounds can be incorporated directly into the support scaffold so that they are slowly released as the support scaffold degrades in vivo. Factors, including nutrients, growth factors, inducers of proliferation or de-differentiation (i.e., causing differentiated cells to lose characteristics of differentiation and acquire characteristics such as proliferation and more general function), products of secretion, immunomodulators, inhibitors of inflammation, regression factors, biologically active compounds which enhance or allow ingrowth of nerve fibers, hyaluronic acid, and drugs, which are known to those skilled in the art and commercially available with instructions as to what constitutes an effective amount, from suppliers such as Collaborative Research and Sigma Chemical Co. Similarly, polymers containing peptides such as the attachment peptide RGD (Arg-Gly-Asp) can be synthesized for use in forming scaffolds (see e.g U.S. Pat. Nos. 4,988,621, 4,792,525, 5,965,997, 4,879,237 and 4,789,734).

In another example, the late passage MSCs cells may be transplanted in a gel scaffold (such as Gelfoam from Upjohn Company), which polymerizes to form a substrate in which the late passage MSCs can grow. A variety of encapsulation technologies have been developed (e.g. Lacy et al., Science 254:1782-84 (1991); Sullivan et al., Science 252:718-712 (1991); WO 91/10470; WO 91/10425; U.S. Pat. No. 5,837,234; U.S. Pat. No. 5,011,472; U.S. Pat. No. 4,892,538). During open surgical procedures, involving direct physical access to the damaged tissue and/or organ, all of the described forms of stem cell delivery preparations are available options. These cells can be repeatedly transplanted at intervals until a desired therapeutic effect is achieved.

### Use of Late Passage MSCs for Small Molecule Transfer

The present invention also provides methods and compositions for delivery of small molecules into a target cell. The method of the invention comprises introducing a small molecule into a donor late passage MSC, and contacting a target cell with a donor cell under conditions permitting the donor cell to form a gap junction with the target cell, whereby the small molecule is delivered into the target cell from the donor late passage MSC The transfer of the small molecule from a late passage MSC to a target cell is via diffusion through gap junctions. The loading of specific small molecules into late passage MSCs can be accomplished by electroporation or by perfusion of late passage MSCs with media containing membrane permeable ester forms. Methods for delivery of small molecules into a target cell, via gap junctions, are disclosed in PCT/US04/042504, which is incorporated by reference herein in its entirety.

Late passage MSCs form gap junction channels with other cells by containing one or more of the following connexins: Cx43, Cx45, Cx40, Cx32 and Cx26. Negatively charged small molecules with minor diameters of about 1.0 nm are all able to transit the aforementioned gap junction channels (homotypic Cx43, Cx40, Cx45, heterotypic Cx43-Cx40 and mixed or heteromeric Cx43-Cx40). The type of gap junctions and total number of channels determine the rate of transit of a specific solute between stem cell and target cell.

Small molecules that are capable of being transferred include, but are not limited to, hydrophilic second messengers, drugs and their metabolites, and inorganic ions. The small molecules may also be oligonucleotides. Such oligonucleotides may be RNA that can traverse the gap junction. The oligonucleotide may be DNA. The oligonucleotide may be an antisense oligonucleotide or a cDNA that produces an antisense oligonucleotide that can traverse the gap junction. The oligonucleotide may be a siRNA oligonucleotide or a cDNA that produces a siRNA oligonucleotide that can traverse the gap junction. The oligonucleotide may be a DNA or RNA that produces a peptide that can traverse the gap junction.

In one embodiment, the invention provides a useful treatment where down regulation of gene expression is desired, for example, with delivery of antisense, ribozyme or siRNA molecules. In a preferred embodiment of the invention, the level of gene expression can be down regulated using antisense or RNAi approaches to inhibit or prevent translation of a gene of interest. Antisense and RNAi approaches involve the design of oligonucleotides (either DNA or RNA) that are complementary to the target MRNA encoding the protein where down regulation is desired. The antisense or siNA oligonucleotides will be targeted to the complementary MRNA transcripts and prevent translation. Absolute complementarity, although preferred, is not required. One skilled in the art can ascertain a tolerable degree of mismatch by use of standard procedures to determine the melting point of the hybridized complex.

In one embodiment, the invention features the use of a double stranded short interfering nucleic acid (siNA) molecule that directs cleavage of the target RNA via RNA interference (RNAi), wherein the double stranded siNA molecule comprises a first and a second strand, each strand of the siNA molecule is about 18 to about 28 nucleotides in length, the first strand of the siNA molecule comprises nucleotide sequence having sufficient complementarity to the target RNA for the siNA molecule to direct cleavage of the target RNA via RNA interference, and the second strand of said siNA molecule comprises nucleotide sequence that is complementary to the first strand.

In yet another embodiment of the invention, ribozyme molecules designed to catalytically cleave target MRNA transcripts can also be used to prevent translation of target mRNA. Alternatively, gene expression can be reduced by targeting deoxyribonucleotide sequences complementary to the regulatory region of the target genes (i.e., promoter and/or enhancers regions) to form triple helical structures that prevent transcription of the target gene. (See generally, Helene, C. et al., 1991, Anticancer Drug Des. 6:569-584 and Maher, L J, 1992, Bioassays 14:807-815).

The oligonucleotides of the invention, i.e., antisense, ribozyme and triple helix forming oligonucleotides, may be synthesized by standard methods known in the art, e.g., by use of an automated DNA synthesizer (such as are commercially available from Biosearch, Applied Biosystems, etc.). Alternatively, recombinant expression vectors may be constructed to direct the expression of the oligonucleotides of the invention. Such vectors can be constructed by recombinant DNA technology methods standard in the art. In a specific embodiment, vectors such as viral vectors may be designed for gene therapy applications where the goal is in vivo expression of inhibitory oligonucleotides in targeted cells.

According to the present invention, a method of delivering a small molecule into a target cell is provided, comprising introducing a small molecule into a donor late passage MSC, and contacting the target cell with the donor late passage MSC or other donor cell under conditions permitting the donor late passage MSC to form a gap junction with the target cell, whereby the small molecule is delivered into the target cell from the donor late passage MSC.

According to the present invention, a method of delivering a small molecule into a syncytial target cell is provided, comprising introducing a small molecule into a donor late passage MSC, and contacting the syncytial target cell with the donor late passage MSC under conditions permitting the donor late passage MSC to form a gap junction with the syncytial target cell, whereby the small molecule is delivered into the syncytial target cell from the donor late passage MSC.

### Use of Late Passage Human Mesenchymal Cells for Generation of Biological Pacemaker Activity

The present invention relates to the generation of biological pacemaker activity based on the expression of wild type, mutant or chimeric HCN ion channels in late passage MSCs for treatment of cardiac disorders. Methods for generating biological pacemaker activity are disclosed in U.S. Pat. No. 6,849,611 and U.S. patent application Ser. Nos. 10/342,506 and 10/757,827 each of which are incorporated by reference herein in their entirety.

As used herein, “biological pacemaker activity” shall mean the rhythmic generation of an action potential originating from the introduction of biological material in a cell or a syncytial structure comprising the cell. A “syncytial structure” shall mean a structure with gap junction-mediated communication between its cells.

The present invention relates to the generation of biological pacemakers with desirable clinical characteristics based on late passage MSCs expression of wild-type, mutant and chimeric HCN genes, and the use of these biological pacemakers to create an effective treatment for cardiac conditions. Accordingly, the present invention provides late passage hMCSs comprising in vitro-recombined gene constructs encoding HCN ion channels. An “HCN ion channel” shall mean a hyperpolarization-activated, cyclic nucleotide-gated ion channel responsible for the hyperpolarization-activated cation currents that are directly regulated by cAMP and contribute to pacemaker activity in heart and brain. “mHCN” designates murine or mouse HCN; “hHCN” designates human HCN.

There are four HCN isoforms: HCN1, HCN2, HCN3 and HCN4. All four isoforms are expressed in brain; HCN1, HCN2 and HCN4 are also prominently expressed in heart, with HCN4 and HCN1 predominating in sinoatrial node and HCN2 in the ventricle.

In an embodiment of the invention, the HCN channel to be expressed is HCN1, HCN2, HCN3, HCN4, or a mutant thereof. Voltage sensing and activation of HCN channels can be altered by mutation. For example, Chen et al. (2001, Proc. Natl. Acad. Sci USA 98:11277-11282) identified three residues, E324, Y331, and R339, in the mHCN2 S4-S5 linker that, when mutated, disrupts normal channel closing. Mutation of a basic residue in the S4 domain (R318Q) prevents channel opening. Conversely, channels with R318Q and Y331S double mutations are constitutively open. Several point mutations, including R318Q, W323A, E324A, E324D, E324K, E324Q, F327A, T330A and Y331A, Y331D, Y331F, Y331K, D332A, M338A, R339A, R339C, R339D, R339E and R339Q, were also made by Chen et al. (2001, Proc. Natl. Acad. Sci USA 98:11277-11282) to investigate in greater detail the role of the E324, Y331 and R339 residues in voltage sensing and activation. Many additional mutations in different HCN isoforms have been reported. For example, Chen et al. (2001, J Gen Physiol 117:491-504) have reported the R538E and R591E mutations in mHCN1; Tsang et al. (2004, J Biol Chem 279:43752-43759) have reported G231A and M232A in mHCN1; Vemana et al (2004, J Gen Physiol 123:21-32) have reported R247C, T249C, K250C, 1251C, L252C, S253C, L254C, L258C, R259C, L260C, S261C, C318S, S338C in mHCN2; Macri and Accili (2004, J Biol Chem 279:16832-16846) have reported S306Q, Y331D AND G404S in mHCN2; and Decher et al. (2004, J Biol Chem 279:13859-13865) have reported Y331A, Y331D, Y331S, R331FD, R339E, R339Q, I439A, S441A, S441T, D443A, D443C, D443E, D443K, D443N, D443R, R447A, R447D, R447E, R447Y, Y449A, Y449D, Y449F, Y449G, Y449W, Y453A, Y453D, Y453F, Y453L, Y453W, P466Q, P466V, Y476A, Y477A and Y481A in mHCN2. The contents of all of the above publications are incorporated herein by reference. Certain of the reported mutations listed above may confer, singly or in combination, beneficial characteristics on the HCN channel with regard to creating a biological pacemaker. The invention disclosed herein encompasses late passage MSC expression of mutations in HCN channels, singly or in combinations, which enhance pacemaker activity of the channel. In a preferred embodiment, the HCN channel or mutant thereof is HCN2.

Mutations are identified herein by a designation with provides the single letter abbreviation of the amino acid residue that underwent mutation, the position of that residue within a polypeptide, and the single letter abbreviation of the amino acid residue to which the residue was mutated. Thus, for example, E324A identifies a mutant polypeptide in which the glutamate residue (E) at position 324 was mutated to alanine (A). Y331A, E324A-HCN2 indicates a mouse HCN2 having a double mutation, one in which tyrosine (Y) at position 331 was mutated to alanine (A), and the other in which the glutamate residue at position 324 was mutated to alanine.

In a specific embodiment of the present invention, the mutant HCN2 channel is E324A-HCN2, Y331A-HCN2, R339A-HCN2, or Y331A,E324A-HCN2. In a preferred embodiment, the mutant HCN2 channel is E324A-HCN2.

One approach to enhancing biological pacemaker activity of a HCN channel by increasing the magnitude of the current expressed and/or speeding its kinetics of activation is to co-express with HCN2 its beta subunit, MiRP1. MiRP1 mutations have also been reported (see e.g., Mitcheson et al., (2000, J Gen Physiol 115:229-40); Lu et al., (2003, J Physiol 551:253-62); Piper et al., (2005, J Biol Chem 280:7206-17)), and certain of these mutations, or combinations thereof, may be beneficial in increasing the magnitude and kinetics of activation of the current expressed by a HCN channel used to create a biological pacemaker. The invention disclosed herein encompasses all such mutations, or combinations thereof, in MiRP1.

The present invention further relates to the use of late passage MSCs expressing chimeras between HCN isoforms for generating pacemaker currents in treating heart disorders. Such chimeric HCN channels may be formed by in vitro recombination of nucleotide sequences encoding portions of all four HCN isoforms to produce HCN chimeras. Chimeras of pacemaker ion channels that may be used in the practice of the invention include, but are not limited to, those chimera channels disclosed in U.S. Provisional Patent Application No. 60/715,934 and 60/832,515, filed Jul. 21, 2006, entitled “Chimeric HCN Channels,” which are both incorporated herein by reference in their entirety.

A “HCN chimera” shall mean an ion channel comprised of portions of more than one type of HCN channel. For example, a chimera of HCN1 and HCN2 or HCN3 or HCN4, and so forth. In an embodiment of the invention, the portions are derived from human HCN isoforms. In addition a chimera ion channel may also comprise portions of an HCN channel derived from different species. For example, one portion of the channel may be derived from a human and another portion may be derived from a non-human.

Such chimeric HCN polypeptides provide an improved characteristic, as compared to a wild-type HCN channel, selected from the group consisting of faster kinetics, more positive activation, increased expression, and/or increased stability, enhanced cAMP responsiveness, and enhanced neurohumoral response.

In general terms, HCN polypeptides can be divided into three major domains: (1) an amino terminal portion; (2) an intramembranous portion and its linking regions; and (3) a carboxy-terminal portion. Structure-function studies have shown that the intramembranous portions with its linking regions play an important role in determining the kinetics of gating. The C-terminal portion contains a binding site for cAMP and so is in large part responsible for the ability of the channel to respond to the sympathetic and parasympathetic nervous systems that respectively raise and lower cellular cAMP levels.

The term “HCNXYZ” (wherein X, Y and Z are any one of the integers 1, 2, 3 or 4, with the proviso that at least one of x, y and Z is a different number from at least one of the remaining) shall mean an HCN chimera channel polypeptide comprising three contiguous portions in the order XYZ wherein X is an N-terminal portion, Y is an intramembrane portion, and Z is a C-terminal portion, and wherein the number of X, Y and Z designates the HCN channel from which that portion is derived. For example, HCN112 is an HCN chimera with a N-terminal portion and intramembrane portion from HCN1 and a C-terminal portion from HCN2.

The present invention provides late passage hMCSs comprising in vitro-recombined gene constructs encoding chimeric HCN channels that have fast kinetics and good responsiveness to cAMP. In one embodiment of the invention described herein, the HCN chimera comprises an amino terminal portion contiguous with an intramembranous portion contiguous with a carboxy terminal portion, wherein each portion is a portion of an HCN channel or a portion of a mutant thereof, and wherein one portion derives from an HCN channel or a mutant thereof which is different from the HCN channel or mutant thereof from which at least one of the other two portions derive.

In a specific embodiment, the mutant HCN channel from which the portion of the HCN chimera derives is E324A-HCN2, Y331A-HCN2, R339A-HCN2, or Y331A,E324A-HCN2. In a still further embodiment, the HCN chimera is a polypeptide comprising mHCN112, mHCN212, mHCN312, mHCN412, mHCN114, mHCN214, mHCN314, mHCN414, hHCN112, hHCN212, hHCN312, hHCN412, hHCN114, hHCN214, hHCN314, or hHCN414. In a specific embodiment of the invention the chimeric HCN polypeptide is hHCN212 or polypeptide mHCN212.

Other preferred embodiments include: a chimeric HCN polypeptide wherein the intramembranous portion is derived from an HCN1 channel; a chimeric HCN polypeptide wherein the intramembranous portion is D140-L400 of hHCN1; or a chimeric HCN polypeptide wherein the intramembranous portion is D129-L389 of mHCN1.

In yet another embodiment of the invention, the chimeric HCN polypeptide is a mutant HCN channel containing a mutation in a region of the channel selected from the group consisting of the S4 voltage sensor, the S4-S5 linker, S5, S6 and S5-S6 linker, the C-linker, and the carboxy-terminal cyclic nucleotide binding domain (“CNBD”).

In yet another embodiment of the invention, the chimeric HCN polypeptide is a mutant, wherein the mutant portion is derived from mHCN2 having the sequence set forth in SEQ ID NO:14 and comprises E324A-mHCN2, Y331A-mHCN2, R339A-mHCN2, or Y331A,E324A-mHCN2. In a specific embodiment of the invention, the mutant portion comprises E324A-mHCN2.

In addition to recombinant expression of wild-type, mutant and chimeric HCN ion channels, the late passage MSCs may further expresses at least one cardiac connexin, including for example, Cx43, Cx40, or Cx45.

To practice the methods of the invention it will be necessary to recombinantly express wild-type, mutant and chimeric HCN ion channels. The cDNA sequence and deduced amino acid sequence of HCN ion channels have been characterized. Sequences of the HCN ion channels are available from public databases.

HCN ion channel nucleotide sequences may be isolated using a variety of different methods known to those skilled in the art. For example, a cDNA library constructed using RNA from a tissue known to express the HCN ion channels can be screened using a labeled HCN channel probe. Alternatively, a genomic library may be screened to derive nucleic acid molecules encoding the HCN ion channel protein. Further, such nucleic acid sequences may be derived by performing a polymerase chain reaction (PCR) using two oligonucleotide primers designed on the basis of known HCN ion channel nucleotide sequences. The template for the reaction may be cDNA obtained by reverse transcription of mRNA prepared from cell lines or tissue known to express the HCN ion channel of interest.

HCN ion channels, polypeptides and peptide fragments, mutated, truncated, deleted and chimeric forms of the HCN channels can be prepared for a variety of uses, including but not limited to, the production of biological pacemaker activity. Such proteins may be advantageously produced by recombinant DNA technology using techniques well known in the art for expressing a nucleic acid. Such methods can be used to construct expression vectors containing the HCN ion channel nucleotide sequences and appropriate transcriptional and translational control signals. These methods include, for example, in vitro recombinant DNA techniques, synthetic techniques, and in vivo genetic recombination. (See, for example, the techniques described in Sambrook J et al. 2000. Molecular Cloning: A Laboratory Manual (Third Edition), and Ausubel et al (1996) Current Protocols in Molecular Biology John Wiley and Sons Inc., USA).

A variety of host-expression vector systems maybe utilized to express the HCN ion channel nucleotide sequences in late passage MSCs. For long-term, high yield production of recombinant HCN ion channel expression, such as that desired for development of biological pacemakers, stable expression is preferred. Rather than using expression vectors which contain origins of replication, host cells can be transformed with DNA controlled by appropriate expression control elements and a selectable marker gene, i.e., tk, hgprt, dhfr, neo, and hygro gene, to name a few. Following the introduction of the foreign DNA, engineered late passage MSCs may be allowed to grow for 1-2 days in enriched media, and then switched to a selective media.

Any of the methods for gene delivery into a host cell available in the art can be used according to the present invention. Such methods include, for example, electroporation, lipofection, calcium phosphate mediated transfection, or viral infection. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215. Exemplary methods are described below.

The present invention further provides compositions comprising MSCs expressing wild-type, mutant or chimera HCN channels, as described above. The compositions of the invention may further comprise a pharmaceutically acceptable carrier.

The present invention relates to a method of treating a subject afflicted with a cardiac rhythm disorder comprising administering a late passage MSC, expressing wild-type, mutant or chimeric HCN polypeptides, to a region of the subject's heart, wherein expression of the HCN polypeptide in said region of the heart is effective to induce a pacemaker current in the heart and thereby treat the subject. In a specific embodiment of the invention, the late passage MSC forms a functional syncytium with the heart.

In an embodiment of the invention, the late passage MSC, expressing wild-type, mutant or chimeric HCN polypeptides is administered to the region of the heart by injection, catheterization, surgical insertion, or surgical attachment. The late passage MSCs may be locally administered by injection or catheterization directly onto or into the heart tissue. The late passage MSCs may be administered by injection or catheterization into at least one of a coronary blood vessel or other blood vessel proximate to the heart. The late passage MSCs may administered to any suitable region of the heart, including, but not limited to, the Bachmanns bundle, sinoatrial node, atrioventricular junctional region, His branch, left or right atrial or ventricle muscle, left or right bundle branch, or Purkinje fibers.

Cardiac rhythm disorders that may be treated using the methods and compositions of the invention include, but are not limited to, sinus node dysfunction, sinus bradycardia, marginal pacemaker function, sick sinus syndrome, tachyarrhythmia, sinus node reentry tachycardia, atrial tachycardia from an ectopic focus, atrial flutter, atrial fibrillation, bradyarrhythmia, or cardiac failure, wherein the late passage MSCs expressing wild-type, mutant or chimeric HCN polypeptides, are administered to the right or left atrial muscle, sinoatrial node, or atrioventricular junctional region of the subject's heart.

Disorders to be treated also include a conduction block, complete atrioventricular block, incomplete atrioventricular block, or bundle branch block, wherein the late passage MSC, expressing wild-type, mutant or chimeric HCN polypeptides, are administered to a region of the subject's heart so as to compensate for the impaired conduction in the heart. Such regions include the ventricular septum or free wall, atrioventricular junctional region, or bundle branch of the ventricle.

The present invention additionally provides a method of inhibiting the onset of a cardiac rhythm disorder in a subject prone to such disorder comprising administering a late passage MSC, expressing wild-type, mutant or chimeric HCN polypeptides, to a region of the subject's heart, wherein expression of the HCN polypeptide in the heart is effective to induce a pacemaker current in the heart and thereby inhibit the onset of the disorder in the subject.

### Use of Late Passage Human Mesenchymal Stem Cells for Generation of a Bypass Bridge

The present invention also provides compositions for treating a subject afflicted with a cardiac rhythm disorder comprising providing a bypass bridge in the heart that will take over the function of a diseased atrioventricular or sinus node. Methods for production of such bypass bridges are disclosed in International Patent Application No. PCT/US04/042953 and U.S. application Ser. No. 11/490,760, filed Jul. 21, 2006, entitled “A Biological Bypass Bridge with Sodium Channels, Calcium Channels and/or Potassium Channels to Compensate for Conduction Block in the Heart,” which are both incorporated herein by reference in their entirety.

In an embodiment of the invention, the bypass bridge may be made from a strip of late passage hMSCs without incorporation of additional molecular determinants of conduction. Here the cells' own ability to generate gap junctions that communicate pacemaker and/or electrical currents/signals are used as a means to propagate an pacemaker and/or electrical wave from cell to cell.

Accordingly, the present invention provides a bypass bridge comprising a tract of gap junction-coupled late passage hMSCs having a first end and a second end, both ends capable of being attached to two selected sites in a heart so as to allow the conduction of an electrical signal across the tract between the two sites, wherein the cells functionally express a sodium channel. Such sodium channels include, for example, a SKM-1 channel which may further comprise an alpha subunit and/or an accessory subunit.

In a specific embodiment of the invention, the first end of the tract is capable of being attached to the atrium and the second end of the tract is capable of being attached to the ventricle, so as to allow conduction of an electrical signal across the tract from the atrium to the ventricle.

In an embodiment of the invention, the late passage MSCs of the bypass bridge may further functionally express a pacemaker ion channel which induces a pacemaker current so as to induce a pacemaker current in said cells. The pacemaker ion channel is at least one of (a) a hyperpolarization-activated, cyclic nucleotide-gated (HCN) ion channel, mutant or chimera thereof, with or without (b) a MiRP1 beta subunit. Mutants and chimeras HCN channels are described in detail above. In an embodiment of the invention, the pacemaker ion channel is expressed in cells in the first end of the tract. In a specific embodiment, the cells expressing the pacemaker ion channel are located in a region extending 0.5 mm from the first end.

The late passage MSCs in the tract may further functionally express one or more additional channels, including but not limited to, a potassium channel which may further comprise a Kir2.1 or Kir2.2 alpha subunit and/or an accessory subunit; and an L-type calcium channel which may further comprise an alpha subunit and an accessory subunit.

Thus, the cells of the bypass bridge may further functionally express one or more of at least one cardiac connexin, an alpha subunit with accessory subunits of an L-type calcium channel, an alpha subunit with or without accessory subunits of a potassium channel, so as to change the voltage-time course of repolarization and/or refractoriness of the heart. Connexins that may be expressed include, but are not limited to, Cx43, Cx40, or Cx45.

The present invention provides a method of making a bypass bridge for implantation in a heart comprising: (a) transfecting a late passage MSC with, and functionally expressing therein, a nucleic acid encoding a sodium channel; and (b) growing the transfected late passage MSC into a tract of cells having a first and a second end capable of being attached to two selected sites in the heart, wherein the cells are physically interconnected via electrically conductive gap junctions.

In an embodiment of the invention, cells in the tract are transfected with a nucleic acid encoding a pacemaker ion channel, wherein the nucleic acid is functionally expressed so as to induce a pacemaker current in the cells. The pacemaker ion channel is at least one of (a) a hyperpolarization-activated, cyclic nucleotide-gated (HCN) ion channel or a mutant or chimera thereof, with or without (b) a MiRP1 beta subunit.

The late passage MSCs may be further transfected with, at least one nucleic acid encoding one or more of at least one cardiac connexin, an alpha subunit with accessory subunits of an L-type calcium channel, an alpha subunit with or without accessory subunits of the potassium channel, such that implantation of a bypass bridge in a heart changes the voltage-time course of repolarization and/or refractoriness of the heart.

The present invention provides a method of implanting a bypass bridge in a heart comprising: (a) making a bypass bridge utilizing the methods of the present invention; (b) selecting a first and a second site in the heart; and (c) attaching the first end of the tract to a first site and the second end of the tract to a second site; so as to thereby implant a bypass bridge in the heart that allows the conduction of a pacemaker and/or electrical signal/current across the tract between the two sites. In an embodiment of the invention, the electrical signal is generated in the atrium by the sinus node or an electronic pacemaker.

The present invention further provides a method of treating a disorder associated with an impaired conduction in a subject's heart comprising: (a) transfecting a late passage MSC with a nucleic acid encoding a sodium channel, wherein the cell functionally expresses the sodium channel; (b) growing the transfected late passage MSC into a tract of cells having a first end and a second end, wherein the cells are physically interconnected via electrically conductive gap junctions; (c) selecting a first site and a second site in the heart between which sites conduction is impaired; and (d) attaching the first end of the tract to the first site and the second end of the tract to the second site; so as to allow the conduction of an electrical signal across the tract between the two sites and thereby treat the subject.

The present invention relates to a method of treating a disorder associated with an impaired conduction and impaired sinus node activity in a subject's heart comprising: (a) transfecting a late passage MSC with at least one nucleic acid encoding a sodium channel and a pacemaker ion channel, wherein the late passage MSC functionally expresses the sodium channel and the pacemaker ion channel; (b) growing the transfected late passage MSC into a tract of cells having a first end and a second end, wherein the cells are physically interconnected via electrically conductive gap junctions;(c) selecting a first site in the left atrium of the heart and a second site, between which sites conduction is impaired; and (d) attaching the first end of the tract to the first site and the second end of the tract to the second site; so as to allow the propagation of an electrical signal generated by the sinus node and/or tract of cells between the two sites and. thereby treat the subject.

The preparation of a bypass bridge in this fashion not only facilitates propagation from atrium to ventricle, but provides sufficient delay from atrial to ventricular contraction to maximize ventricular filling and emptying to mimic the normal activation and contractile sequence of the heart. Moreover, this approach, when used with biological pacemaker technology to improve atrial impulse initiation in the setting of sinus node disease offers a completely physiologic system. Thus, the present methods comprise the use in a subject's heart of various combinations of a biological pacemaker and/or biological atrioventricular bridge or atrioventricular node.

### Use of Mesenchymal Stem Cells in Biological Pacemakers and/or Bypass Bridges in Tandem with Electronic Pacemakers

The present invention relates to the use of MSCs in biological pacemakers and/or bypass bridges either alone or in combination with electronic pacemakers. Detailed descriptions of the individual components of a tandem pacemaker have been previously published. For example, details of electronic pacemakers per se may be found in U.S. Pat. No. 5,983,138; U.S. Pat. No. 5,318,597; U.S. Pat. No. 5,376,106; Pacemaker Timing Cycles and Electrocardiography, David L. Hayes, M.D., Chapter 6 of Cardiac Pacing and Defibrillation, pp. 201-223, Mayo Foundation, 2000; and Types of Pacemakers and Hemodynamics of Pacing, Chapter 5 of A Practical Guide to Cardiac Pacing-Fifth Edition, pp. 78-84, Cippincott Williams & Wilkins, Philadelphia (2000) all of which are incorporated herein by reference. Additionally, tandem cardiac pacemakers to be used in combination with biological pacemakers and/or bypass bridges are described in U.S. patent application Ser. Nos. 60/701,312 (filed on Jul. 21, 2005) and 60/781,723 (filed on Mar. 14, 2005) and 11/490,997 (filed on Jul. 21, 2006), entitled “Tandem pacemaker systems” each of which are incorporated by reference herein in their entirety.

In preferred embodiments of the subject invention, the electronic pacemaker is programmed to produce its pacemaker signal on an “as-needed” basis, i.e., to sense the biologically generated beats and to discharge electrically when there has been failure of the biological pacemaker to fire and/or atrioventricular bridge to conduct an impulse for more than a preset time interval. At this point the electronic pacemaker will take over the pacemaker function until the biological pacemaker resumes activity and/or the atrioventricular bridge resumes impulse conduction. Accordingly, a determination should be made on when the electronic pacemaker will produce its pacemaker signal. State of the art pacemakers have the ability to detect when the heart rate falls below a threshold level in response to which an electronic pacemaker signal should be produced. The threshold level may be a fixed number, but preferably it varies depending on patient activity such as physical activity or emotional status. When the patient is at rest or pursuing light activity the patient's baseline heart rate may be at 50-80 beats per minute (bpm) (individualized for each patient), for example. Of course, this baseline heart rate varies depending on the age and physical condition of the patient, with athletic patients typically having lower baseline heart rates. The electronic pacemaker can be programmed to produce a pacemaker signal when the patient's actual heart rate (including that induced by any biological pacemaker) falls below a certain threshold baseline heart rate, a certain differential, or other ways known to those skilled in the art. When the patient is at rest the baseline heart rate will be the resting heart rate. The baseline heart rate will likely change depending on the physical activity level or emotional state of the patient. For example, if the baseline heart rate is 80 bpm, the electronic pacemaker may be set to produce a pacemaker signal when the actual heart rate is detected to be about 64 bpm (i.e., 80% of 80 bpm).

The electronic component can also be programmed to intervene at times of exercise if the biological component fails, by intervening at a higher heart rate and then gradually slowing to a baseline rate. For example, if the heart rate increases to 120 bpm due to physical activity or emotional state, the threshold may increase to 96 bpm (80% of 120 bpm). The biological portion of this therapy brings into play the autonomic responsiveness and range of heart rates that characterize biological pacemakers and the baseline rates that function as a safety-net, characterizing the electronic pacemaker. The electronic pacemaker may be arranged to output pacemaker signals whenever there is a pause of an interval of X% (e.g., 20%) greater than the previous interval, as long as the previous interval was not due to an electronic pacemaker signal and was of a rate greater than some minimum rate (e.g., 50 bpm).

In an embodiment of the present methods, the electronic pacemaker senses the heart beating rate and produces a pacemaker signal when the heart beating rate falls below a specified level. In a further embodiment, the specified level is a specified proportion of the beating rate experienced by the heart in a reference time interval. In a still further embodiment, the reference time interval is an immediately preceding time period of specified duration.

The present invention provides a tandem pacemaker system comprising (1) an electronic pacemaker, and (2) a biological pacemaker, wherein the biological pacemaker comprises an implantable late passage MSC that functionally expresses a wild type, mutant or chimeric hyperpolarization-activated, cyclic nucleotide-gated (HCN) ion channel, and wherein the expressed HCN channel generates an effective pacemaker current when the cell is implanted into a subject's heart. Wild type, mutant and chimeric HCN channel expression can be achieved using the methods described above.

In an embodiment of the invention, the biological pacemaker of the tandem system comprises at least about 5,000 late passage MSCs. In another embodiment of the invention, the biological pacemaker comprises at least about 200,000 late passage MSCs. In another embodiment of the invention, the biological pacemaker comprises at least about 700,000 late passage MSCs.

In a specific embodiment of the invention, a tandem pacemaker system is provided comprising (1) an electronic pacemaker, and (2) a biological pacemaker, wherein the biological pacemaker comprises an implantable late passage MSC, wherein said cell functionally expresses a chimeric HCN ion channel, wherein said chimeric HCN is hHCN212, and wherein the expressed chimeric HCN channel generates an effective pacemaker current when the cell is implanted into a subject's heart, and wherein the biological pacemaker comprises at least about 700,000 human adult mesenchymal late passage MSCs.

Further, the present invention provides a tandem pacemaker system comprising (1) an electronic pacemaker, and (2) a bypass bridge comprising a strip of gap junction-coupled late passage MSCs having a first end and a second end, both ends capable of being attached to two selected sites in a heart, so as to allow the transmission of a pacemaker and/or electrical signal/current across the tract between the two sites in the heart.

In a specific embodiment of the invention, the first end of the bypass bridge is capable of being attached to the atrium and the second end capable of being attached to the ventricle, so as to allow transmission of an electrical signal from the atrium to travel across the tract to excite the ventricle. Further, the late passage MSCs of the bypass bridge can functionally express at least one protein selected from the group consisting of: a cardiac connexin; an alpha subunit and accessory subunits of a L-type calcium channel; an alpha subunit with or without the accessory subunits of a sodium channel; and a L-type calcium and/or sodium channel in combination with the alpha subunit of a potassium channel, with or without the accessory subunits of the potassium channel. Such cardiac connexins are selected from the group consisting of Cx43, Cx40, and Cx45.

Further, the present invention provides a tandem pacemaker system comprising (1) an electronic pacemaker, (2) a bypass bridge comprising a strip of gap junction-coupled late passage MSCs having a first end and a second end, both ends capable of being attached to two selected sites in a heart, so as to allow the transmission of a pacemaker and/or electrical signal/current across the tract between the two sites in the heart, and (3) a biological pacemaker comprising comprises an implantable late passage MSC that functionally expresses a (a) an HCN ion channel, or (b) a chimeric HCN channel wherein the chimeric HCN channel comprises portions of more than one type of HCN channel, or (c) a mutant HCN channel wherein the expressed HCN, chimeric HCN or mutant HCN channel generates an effective pacemaker current when said cell is implanted into a subject's heart. In an embodiment of the invention, the biological pacemaker of the tandem system, comprises at least about 5,000 late passage MSCs. In another embodiment of the invention, the biological pacemaker comprises at least about 200,000 late passage MSCs. In another embodiment of the invention, the tandem pacemaker system comprises at least about 700,000 late passage MSCs.

The present invention provides a method of treating a subject afflicted with a cardiac rhythm disorder, which method comprises administering a tandem pacemaker system as described herein to the subject, wherein the biological pacemaker of the system is provided to the subject's heart to generate an effective biological pacemaker current and further providing the electronic pacemaker to the subject's heart to work in tandem with the biological pacemaker to treat the cardiac rhythm disorder. The electronic pacemaker may be provided before the biological pacemaker, simultaneously with the biological pacemaker or after the biological pacemaker. The biological pacemaker is designed to enhance beta-adrenergic responsiveness of the heart, decreases outward potassium current I_{K1}, and/or increases inward current I_{f}.

Further, the biological pacemaker may be provided to the Bachman's bundle, sinoatrial node, atrioventricular junctional region, His branch, left or right bundle branch, Purkinke fibers, right or left atrial muscle or ventricular muscle of the subject's heart.

Cardiac rhythm disorders that may be treated using the tandem systems of the invention include, for example, sinus node dysfunction, sinus bradycardia, marginal pacemaker activity, sick sinus syndrome, tachyarrhythmia, sinus node reentry tachycardia, atrial tachycardia from an ectopic focus, atrial flutter, atrial fibrillation, bradyarrhythmia, or cardiac failure and wherein the biological pacemaker is administered to the left or right atrial muscle, sinoatrial node, or atrioventricular junctional region of the subject's heart.

In an embodiment of the invention, the electronic pacemaker is programmed to sense the subject's heart beating rate and to produce a pacemaker signal when the heart beating rate falls below a selected heart beating rate. The selected beating rate is a selected proportion of the beating rate experienced by the heart in a reference time interval. The reference time interval is an immediately preceding time period of selected duration.

The present invention provides a method of treating a cardiac rhythm disorder, wherein the disorder is a conduction block, complete atrioventricular block, incomplete atrioventricular block, bundle branch block, cardiac failure, or a bradyarrhythmia, the method comprising administering a tandem pacemaker system comprising a bypass tract and an electronic pacemaker to the subject's heart such that the bypass tract spans the region exhibiting defective conductance, wherein transmission by the bypass tract of an electronic pacemaker current induced by the electronic pacemaker is effective to treat the subject, and wherein the electronic pacemaker is provided either prior to, simultaneously with or after the bypass tract is provided.

The present invention is also directed to a method of treating a subject afflicted with a sinus node dysfunction, sinus bradycardia, marginal pacemaker activity, sick sinus syndrome, cardiac failure, tachyarrhythmia, sinus node reentry tachycardia, atrial tachycardia from an ectopic focus, atrial flutter, atrial fibrillation, or a bradyarrhythmia and a conduction block disorder, which method comprises administering a tandem pacemaker system comprising a biological pacemaker, a bypass tract and an electronic pacemaker, wherein an electronic pacemaker is provided either prior to, simultaneously with, or after the biological pacemaker is provided, and wherein the biological pacemaker is administered to the subject to generate an effective biological pacemaker current in the subject's heart, and wherein a bypass tract spans the region exhibiting defective conduction, wherein transmission by the bypass tract of an electronic pacemaker and/or biological pacemaker current is effective to treat the subject.

The present invention further relates to a method of treating a subject afflicted with ventricular dyssynchrony comprising (a) selecting a site in a first ventricle of the subject's heart, (b) administering a biological pacemaker of as described herein to the selected site so as to initiate pacemaker activity and stimulate contraction of the first ventricle, and (c) pacing a second ventricle of the heart with a first electronic pacemaker which is programmed to detect a signal from the biological pacemaker and to produce a pacemaker signal at a reference time interval after the biological pacemaker signal is detected, thereby providing biventricular pacemaker function to treat the subject.

In a specific embodiment, the electronic pacemaker is further programmable to produce a pacemaker signal when it fails to detect a signal from the biological pacemaker after a time period of specified duration. Additionally, the system may further comprise a second electronic pacemaker to be administered to a coronary vein, wherein the second electronic pacemaker is programmable to detect a signal from the biological pacemaker and to produce a pacemaker signal in tandem with the first electronic pacemaker if said second electronic pacemaker fails to detect a signal from the biological pacemaker after a time period of specified duration, the first and second electronic pacemakers thereby providing biventricular function.

A tandem pacemaker system for treating a subject afflicted with ventricular dyssynchrony is provided comprising (1) a biological pacemaker to be administered to a first ventricle of the subject's heart, and (2) an electronic pacemaker to be administered to a second ventricle of the subject's heart, wherein the electronic pacemaker is programmable to detect a signal from the biological pacemaker and to produce a electronic pacemaker signal at a reference time interval after the biological pacemaker signal is detected, so as to thereby provide biventricular pacemaker function, and wherein the electronic pacemaker is provided either prior or simultaneously with the biological pacemaker.

Such a pacemaker system may further comprise a second electronic pacemaker to be administered to a coronary vein, wherein the second electronic pacemaker is programmable to detect a signal from the biological pacemaker and to produce a pacemaker signal in tandem with the first electronic pacemaker if said second electronic pacemaker fails to detect a signal from the biological pacemaker after a time period of specified duration, the first and second electronic pacemakers thereby providing biventricular function.

### Uses and Administration of the Compositions of the Invention

The present invention provides methods and compositions which may be used for treatment of various diseases associated with cardiac disorders. The term “cardiac disorder” as used herein refers to diseases that result from any impairment in the heart's pumping function. This includes, for example, impairments in contractility, impairments in ability to relax (sometimes referred to as diastolic dysfunction), abnormal or improper functioning of the heart's valves, diseases of the heart muscle (sometimes referred to as cardiomyopathy), diseases such as angina pectoris and myocardial ischemia and infarction characterized by inadequate blood supply to the heart muscle, infiltrative diseases such as amyloidosis and hemochromatosis, global or regional hypertrophy (such as may occur in some kinds of cardiomyopathy or systemic hypertension), and abnormal communications between chambers of the heart (for example, atrial septal defect). For further discussion, see Braunwald, Heart Disease: a Textbook of Cardiovascular Medicine, 5th edition, W B Saunders Company, Philadelphia Pa. (1997) (hereinafter Braunwald). The term “cardiomyopathy” refers to any disease or dysfunction of the myocardium (heart muscle) in which the heart is abnormally enlarged, thickened and/or stiffened. As a result, the heart muscle's ability to pump blood is usually weakened. The disease or disorder can be, for example, inflammatory, metabolic, toxic, infiltrative, fibroplastic, hematological, genetic, or unknown in origin. There are two general types of cardiomyopathies: ischemic (resulting from a lack of oxygen) and nonischemic. Other diseases include congenital heart disease which is a heart-related problem that is present since birth and often as the heart is forming even before birth or diseases that result from myocardial injury which involves damage to the muscle or the myocardium in the wall of the heart as a result of disease or trauma. Myocardial injury can be attributed to many things such as, but not limited to, cardiomyopathy, myocardial infarction, or congenital heart disease. Specific cardiac disorders to be treated also include congestive heart failure, ventricular or atrial septal defect, congenital heart defect or ventricular aneurysm. The cardiac disorder may be pediatric in origin. The cardiac disorder may require ventricular reconstruction.

Cardiac rhythm disorders that may be treated include pathological arrhythmia, conduction block, complete atrioventricular block, incomplete atrioventricular block, bundle branch block, weak pacemaker activity, sinus node dysfunction, sinus bradycardia, sick sinus syndrome, bradyarrhythmia, tachyarrhythmia, Sinoatrial nodal re-entry tachycardia, atrial tachycardia from an ectopic focus, atrial flutter, atrial fibrillation, or cardiac failure.

The methods of the invention, comprise administration of late passage MSCs in a pharmaceutically acceptable carrier, for treatment of cardiac disorders. “Administering” shall mean delivering in a manner which is effected or performed using any of the various methods and delivery systems known to those skilled in the art. Administering can be performed, for example, pericardially, intracardially, subepicardially, transendocardially, via implant, via catheter, intracoronarily, intravenously, intramuscularly, subcutaneously, parenterally, topically, orally, transmucosally, transdermally, intradermally, intraperitoneally, intrathecally, intralymphatically, intralesionally, epidurally, or by in vivo electroporation. Administering can also be performed, for example, once, a plurality of times, and/or over one or more extended periods.

Cell-based biological pacemaker may require focal delivery. Several methods to achieve focal delivery are feasible; for example, the use of catheters and needles, and/or growth on a matrix and a “glue.” Whatever approach is selected, the delivered cells should not disperse from the target site. Such dispersion could introduce unwanted electrical effects within the heart or in other organs.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. The composition can be formulated as a suppository, with traditional binders and carriers such as triglycerides. Oral formulation can include standard carvers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical sciences” by E. W. Martin. Such compositions will contain a therapeutically effective amount of the therapeutic compound, preferably in purified form, together with a suitable amount of carrier so as to provide the form for proper administration to the patient. The formulation should suit the mode of administration.

The appropriate concentration of the composition of the invention which will be effective in the treatment of a particular cardiac disorder or condition will depend on the nature of the disorder or condition, and can be determined by one of skill in the art using standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses maybe extrapolated from dose response curves derived from in vitro or animal model test systems. Additionally, the administration of the compound could be combined with other known efficacious drugs if the in vitro and in vivo studies indicate a synergistic or additive therapeutic effect when administered in combination.

The progress of the recipient receiving the treatment may be determined using assays that are designed to test cardiac function. Such assays include, but are not limited to ejection fraction and diastolic volume (e.g., echocardiography), PET scan, CT scan, angiography, 6-minute walk test, exercise tolerance and NYHA classification.

### EXAMPLE

### Biological Features of Late Passage Mesenchymal Stem Cells

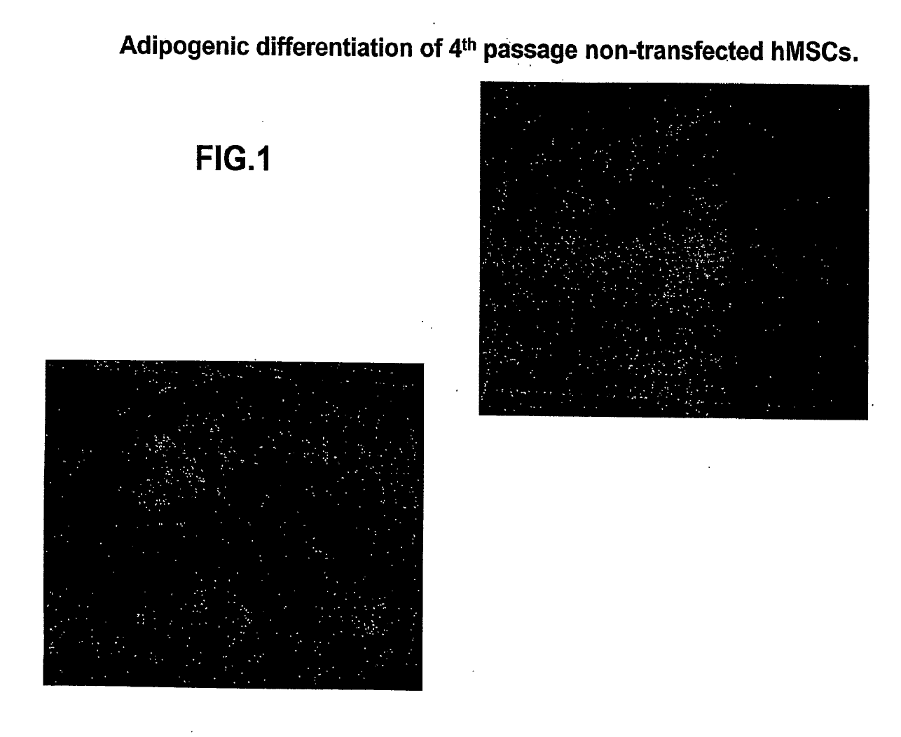

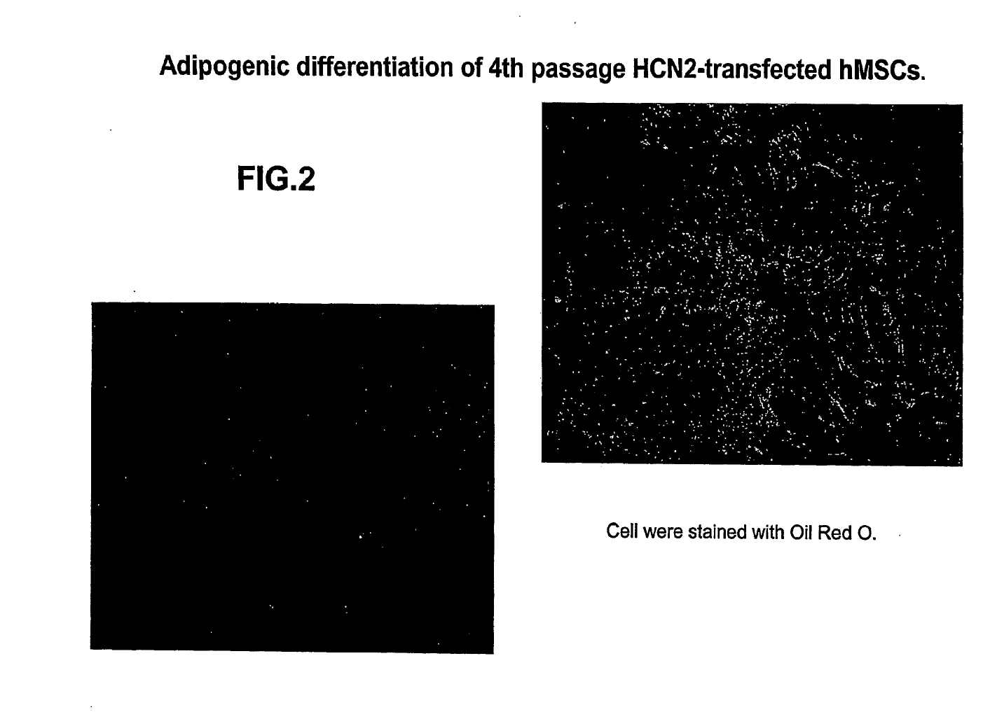

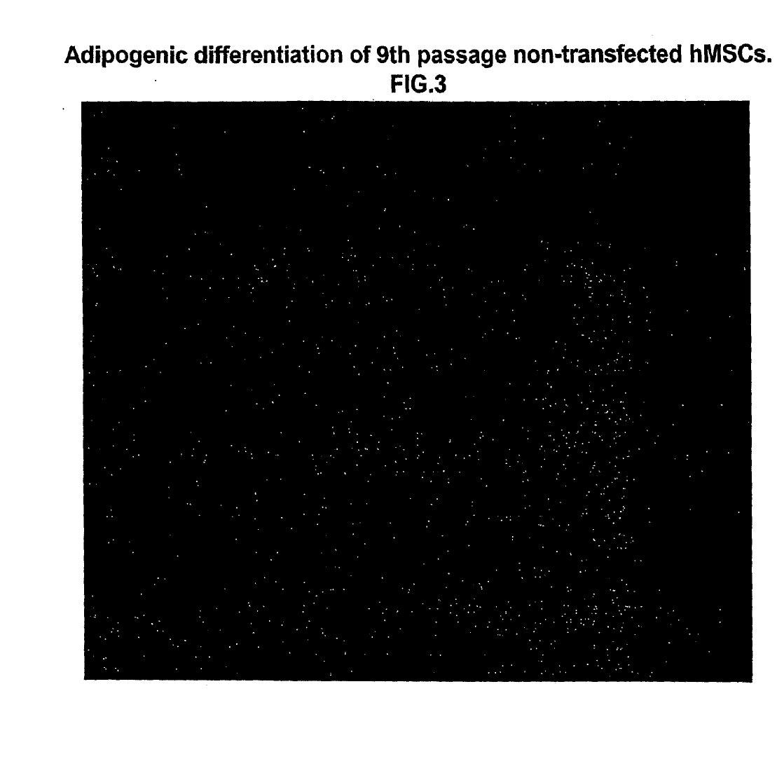

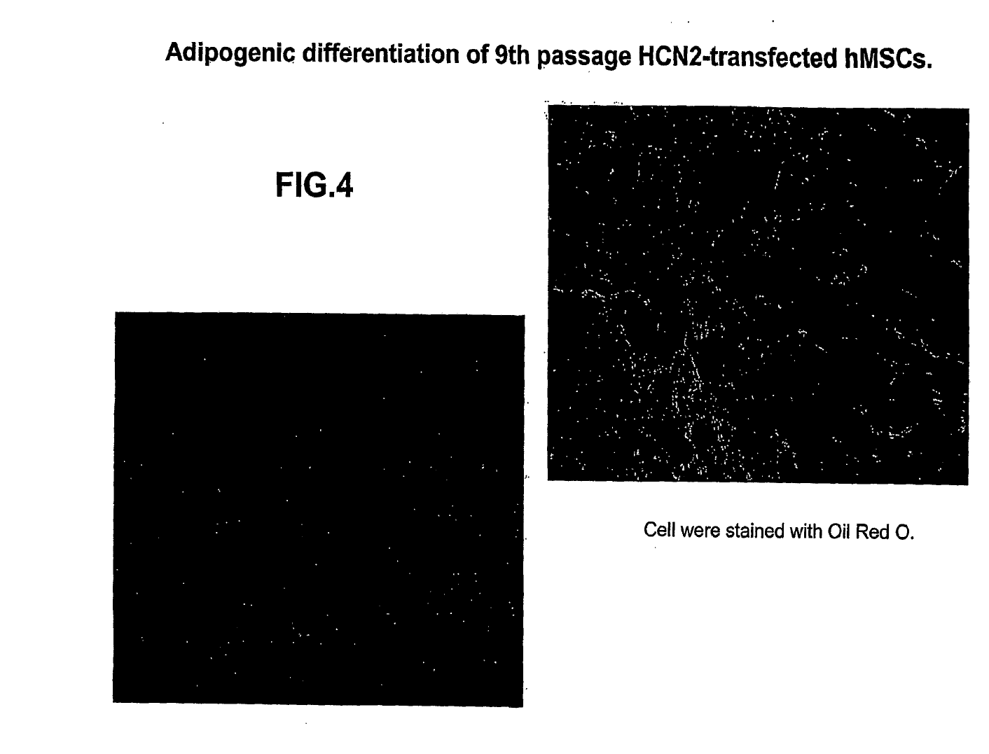

Experiments were performed to determine the biological features of late passage MSCs. hMSCs were purchased and thawed, subcultured and maintained according to the supplier's directions (Cambrex Corporation.). As demonstrated in FIG. 1, fat vacuoles are observed in 4^{th} passage hMSCs exposed to adipogenic differentiation using a purchased kit and the manufacturer's directions (see instructions for adipogenic assay procedure from Cambrex Corporation). In 4^{th} passage hSCs first transfected with the PIRES-HCN2 plasmid followed by exposure to adipogenic differentiation, fewer cells with fat vacuoles were observed, but staining with oil red O still demonstrates a significant number of positive (red) cells (FIG. 2). See instructions for oil red O staining for in vitro adipogenesis from Cambrex Corporation. In contrast, minimal adipogenic differentiation of 9^{th} passage non-transfected hMSCs is demonstrated by the presence of few fat vacuoles (FIG. 3). FIG. 4 indicates the absence of adipogenic differentiation in 9^{th} passages hMSCs transfected with the PIRES-HCN2 plasmid.

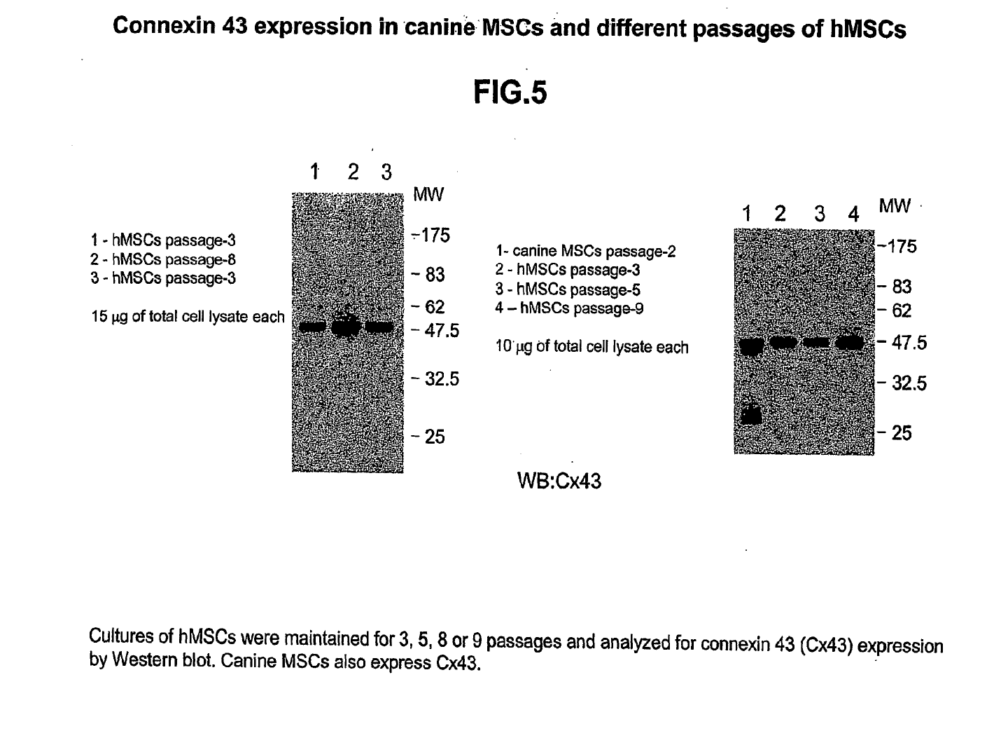

FIG. 5 depicts Western blots demonstrating abundant connexin 43 expression in 3^{rd} and 8^{th} passage hMSCs (right panel) and 3, 5 and 9^{th} passage hMSCs and 2^{nd} passage canine hMSCs (right panel).

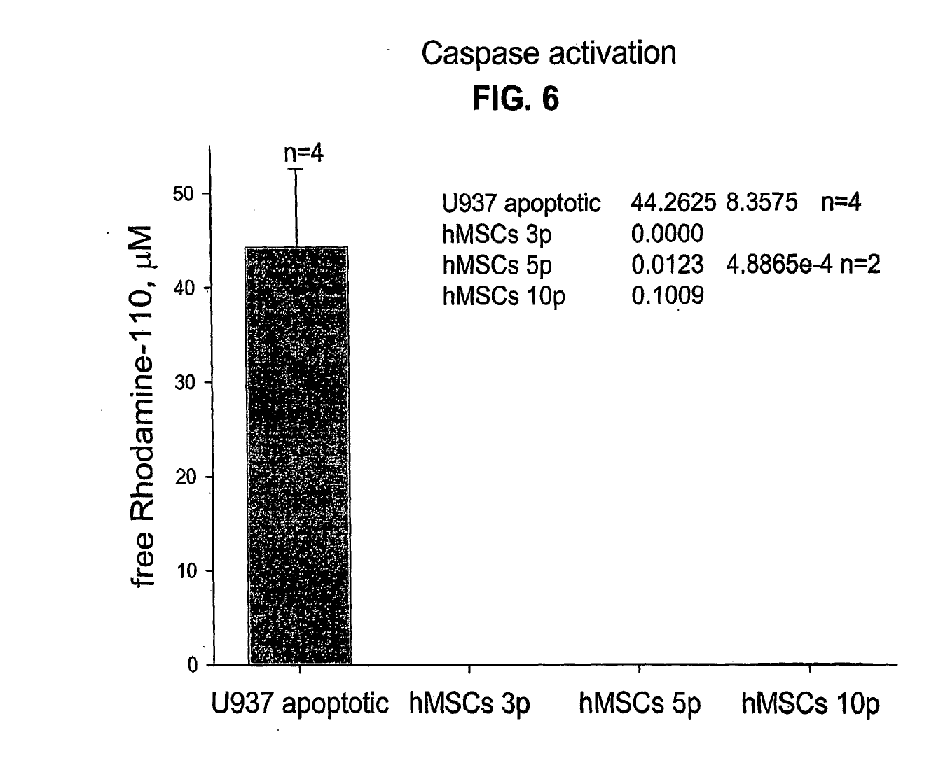

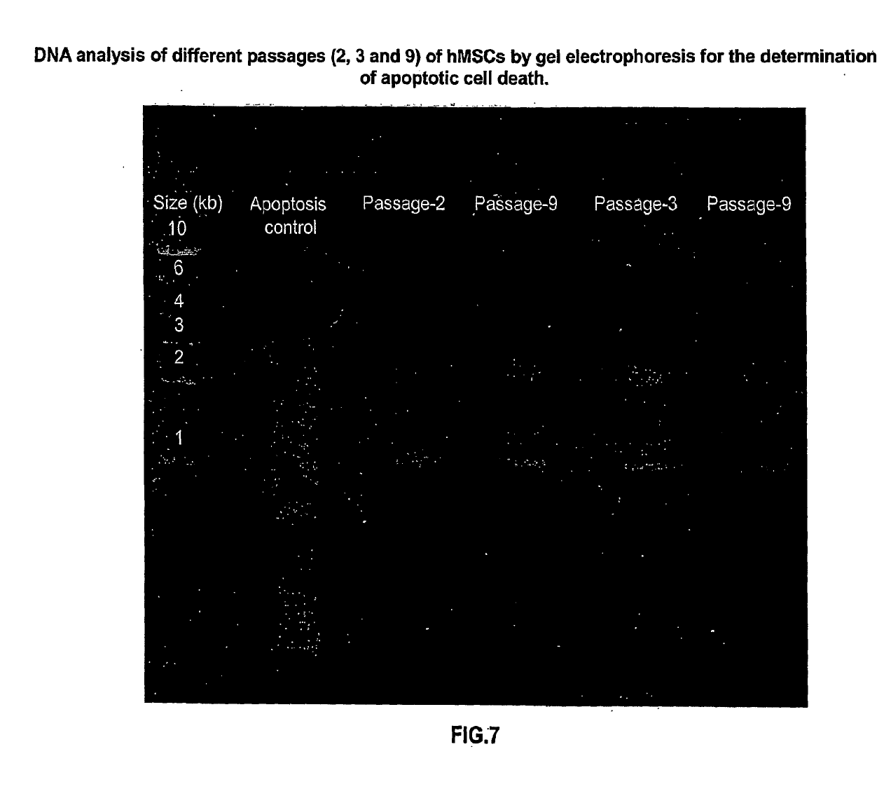

To determine the predisposition of late passage MSCs to apoptosis, caspase activation was assayed for. FIG. 6 demonstrates minimal activation for hMSCs at passages 3, 5 or 10 indicating no predisposition to apoptosis. Additionally, as depicted in FIG. 7. there is no DNA fragmentation, further indicating that these passaged hMSCs do not have a predisposition to apoptosis.

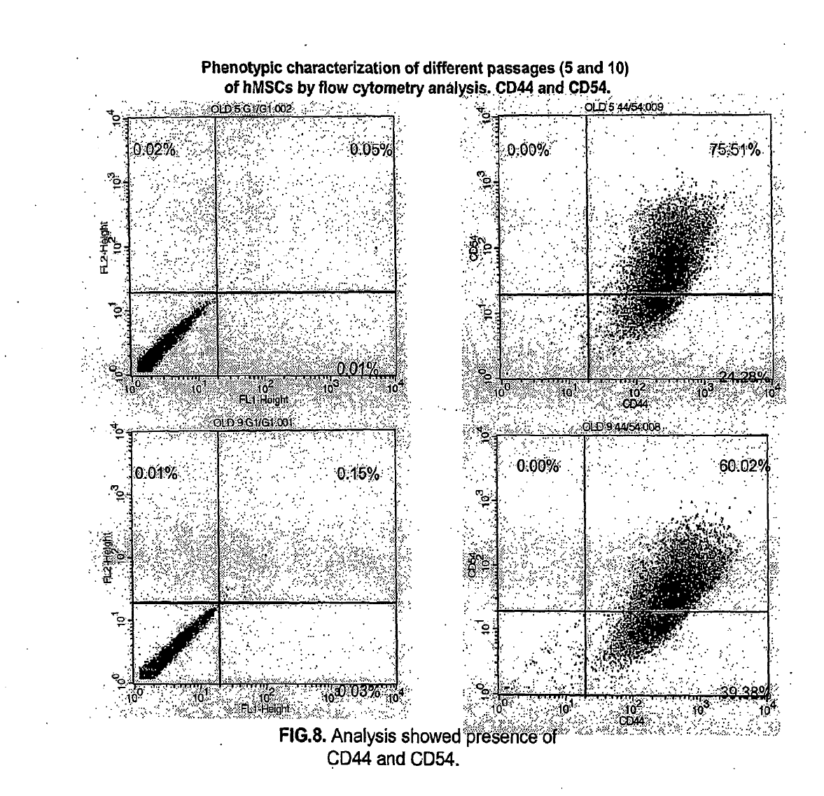

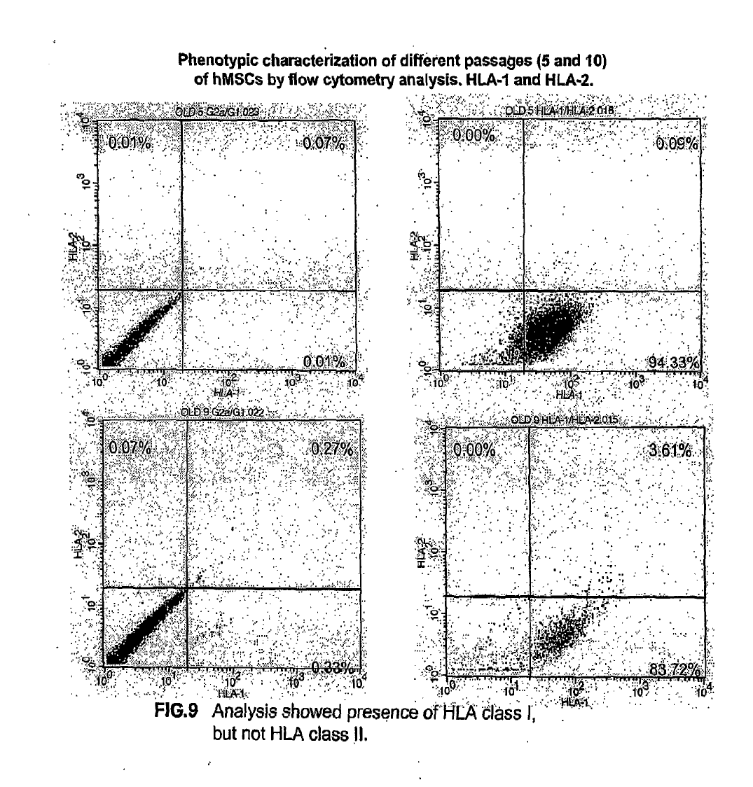

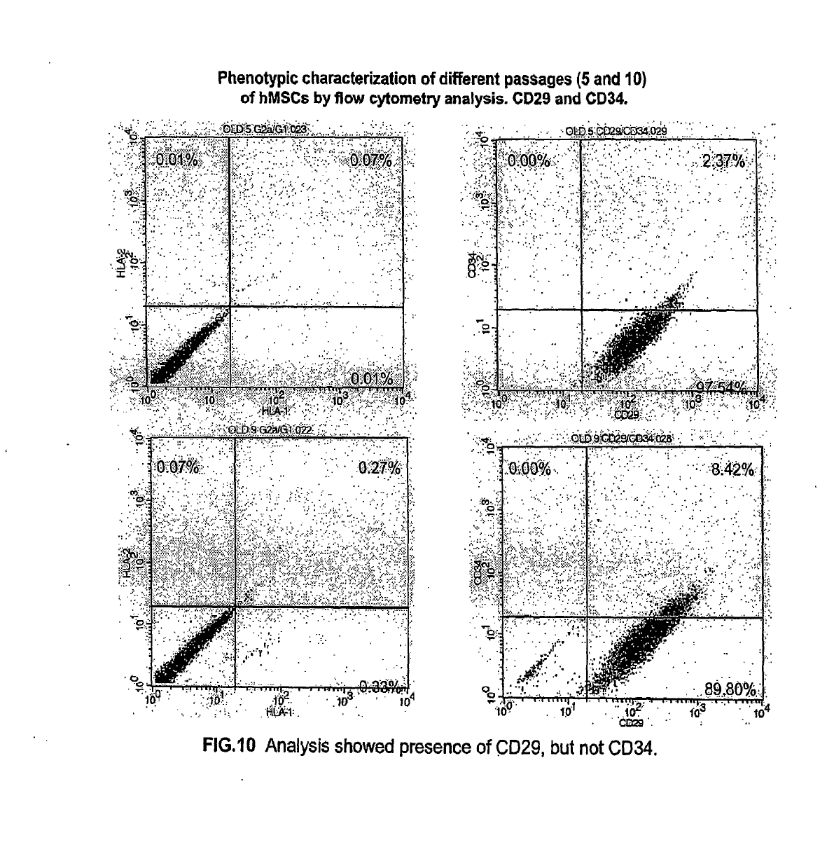

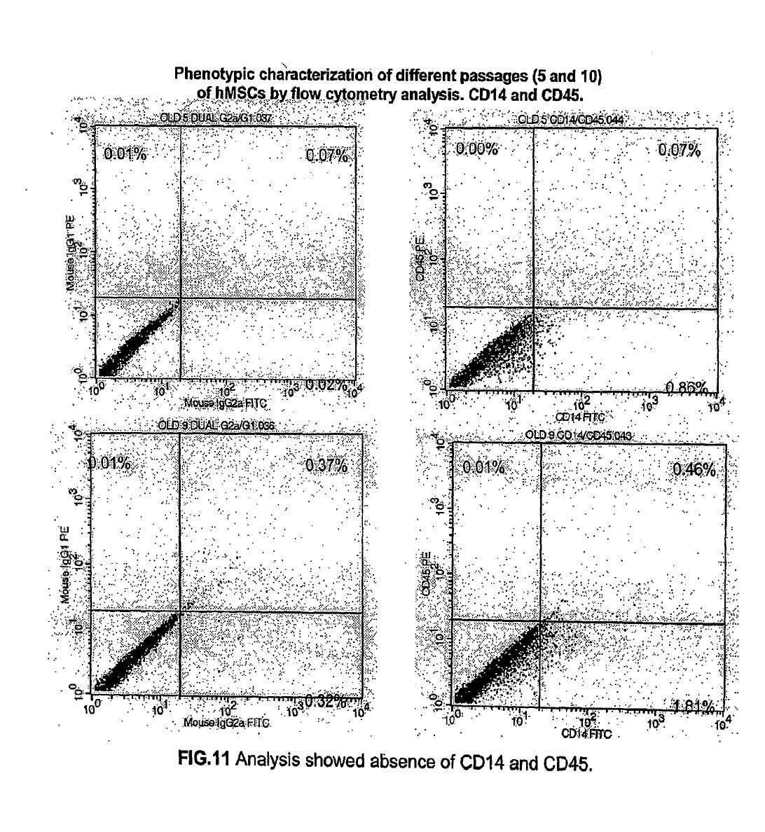

Phenotypic characterization of cell surface antigen expression was examined on late passage MSCs by flow cytometry. The results indicate the presence of CD44 and CD54 antigen (FIG. 8), the presence of HLA I markers but not HLA class II markers (FIG. 9) and the presence of CD29 but not CD34 in both passage 5 and 10 cells. FIG. 11 demonstrates the absence of CD14 and CD45 antigens in both sets of cells.

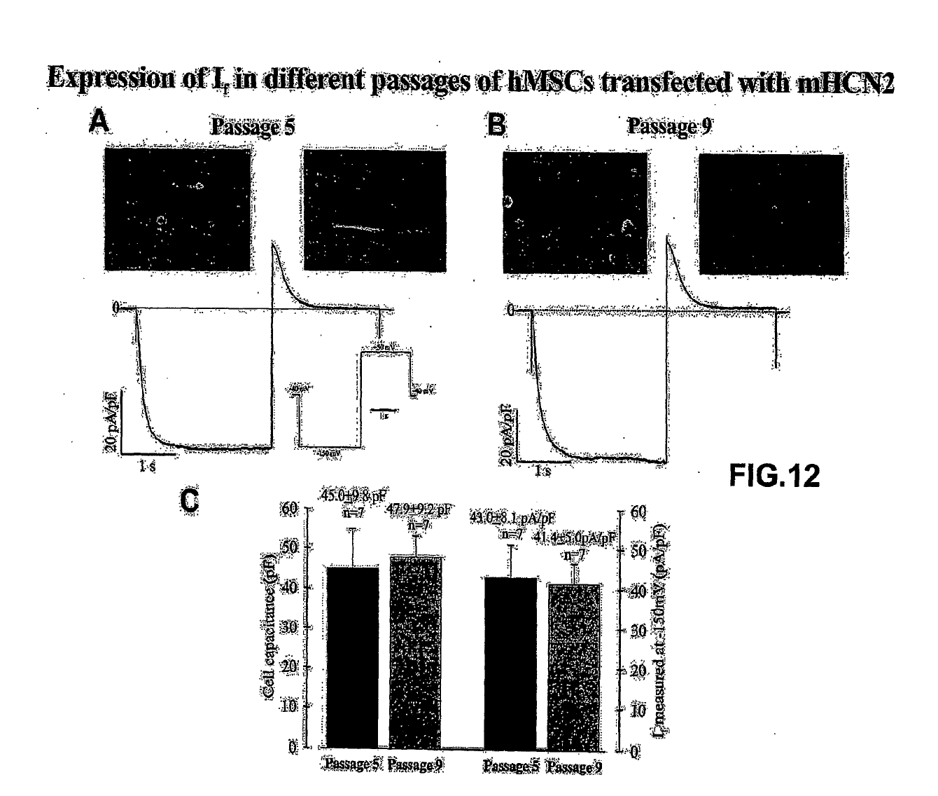

FIG. 12 demonstrates that expression of HCN2-induced I_{f} like current is the same in cells from passages 5 and 9 transfected with the PIRES-HCN2 plasmid: FIG. 12A depicts fluorescence images of passage 5 cells (upper two panels) and sample current record from patch clamp recordings (lower panel). FIG. 12B depicts fluorescence images of passage 9 cells (upper 2 panels) and sample current record from patch clamp recordings (lower panel); FIG. 12C is a histogram comparing the capacitance (left 2 bars) and the HCN2-induced current density (right two bars). There is no significant difference in either parameter between hMSCs from passage 5 and 9.

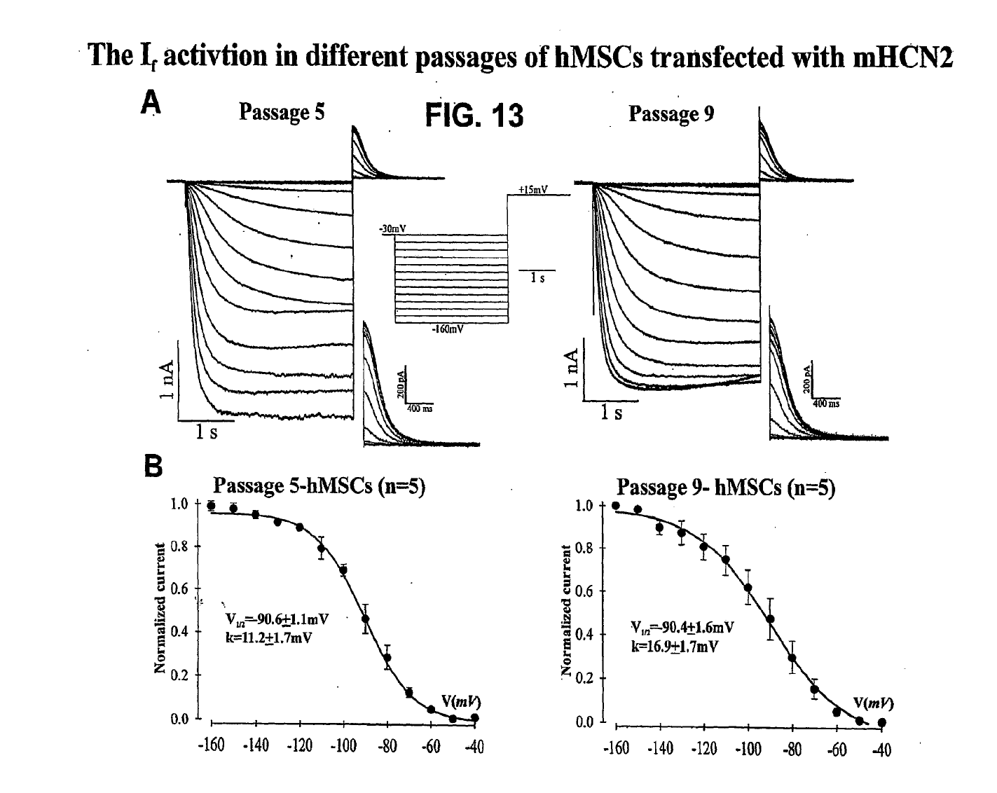

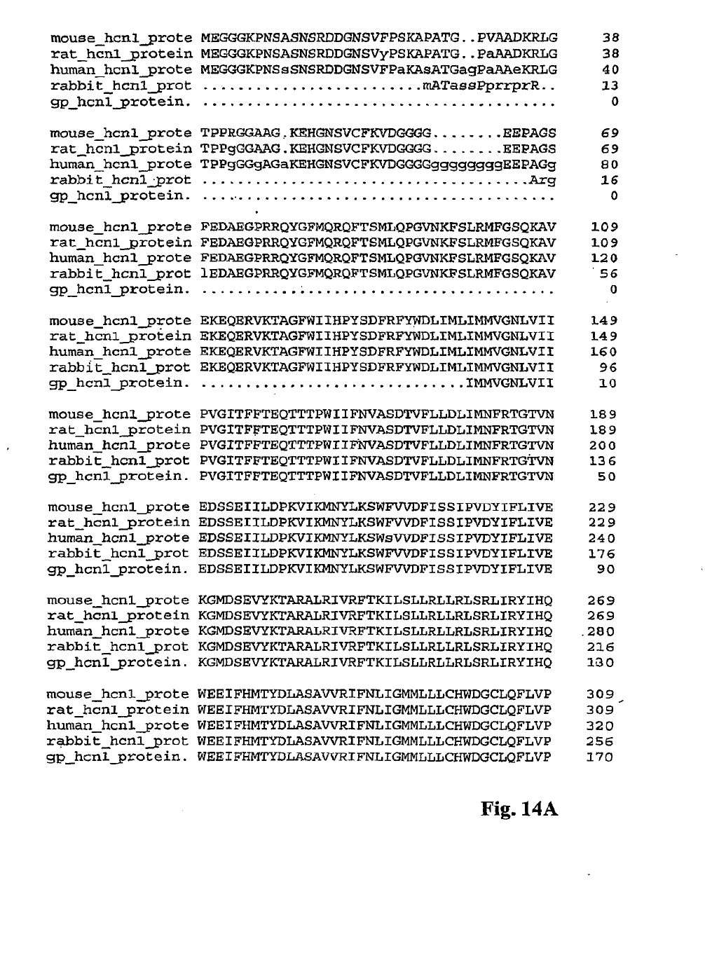

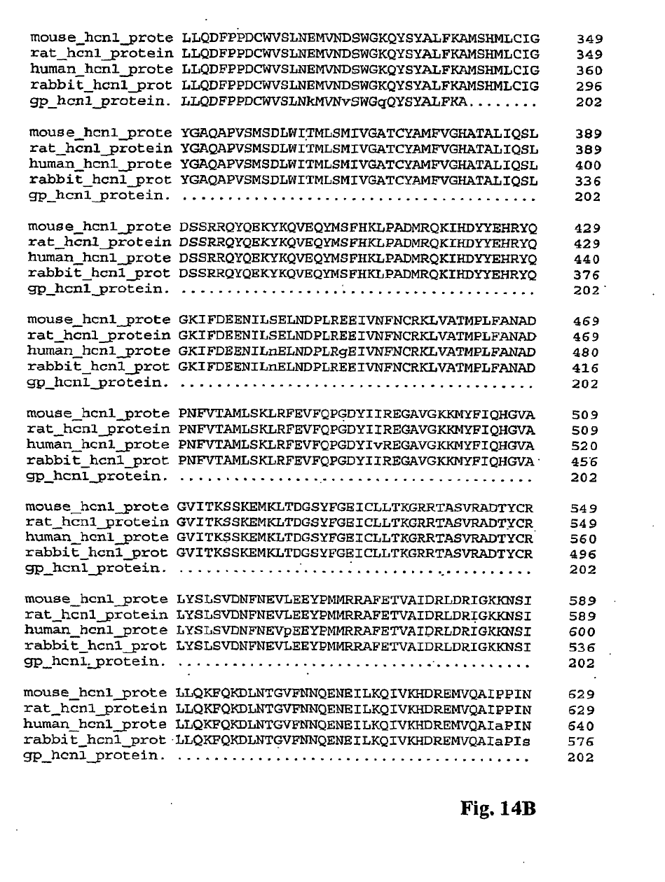

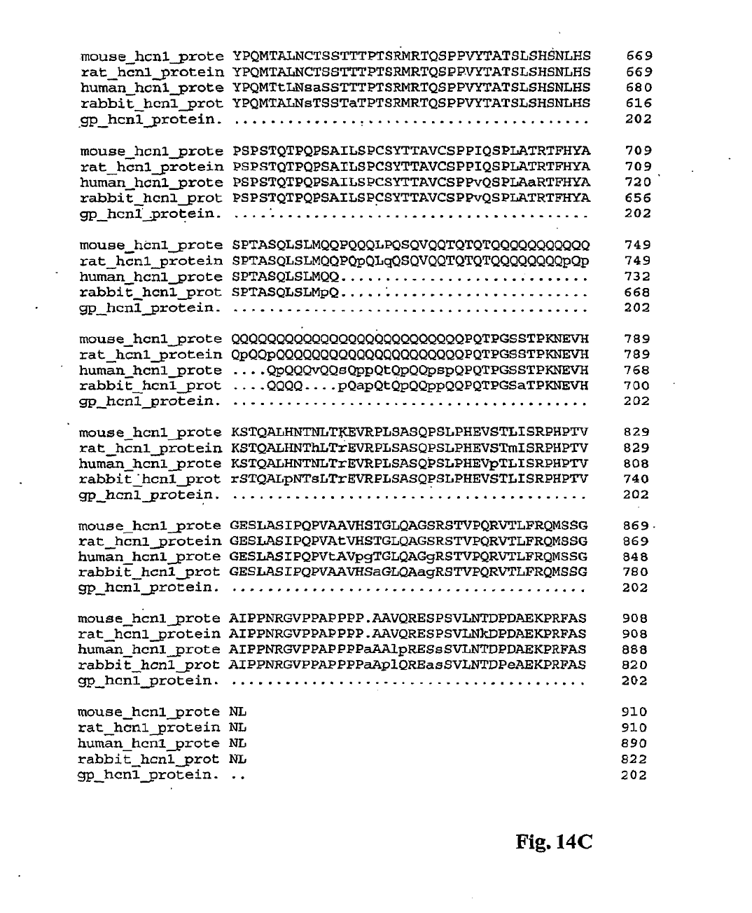

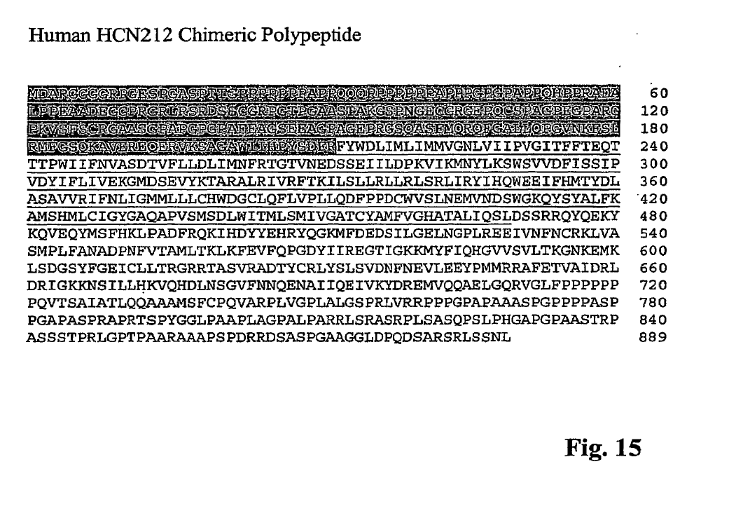

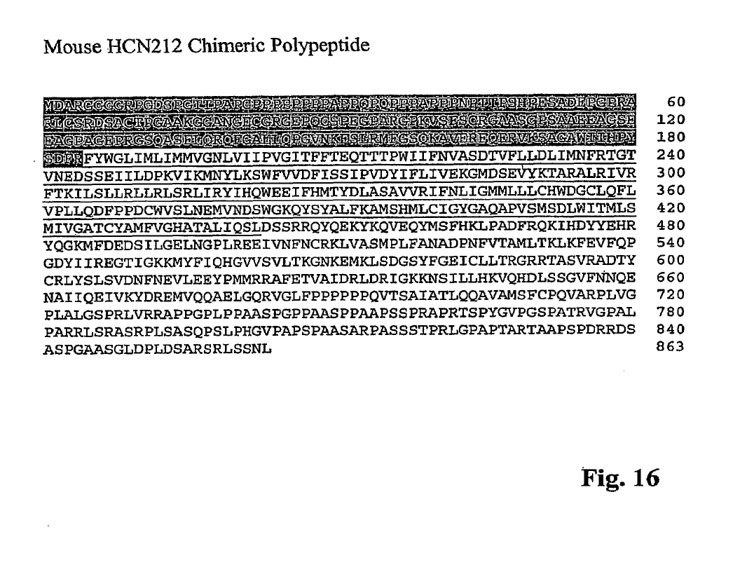

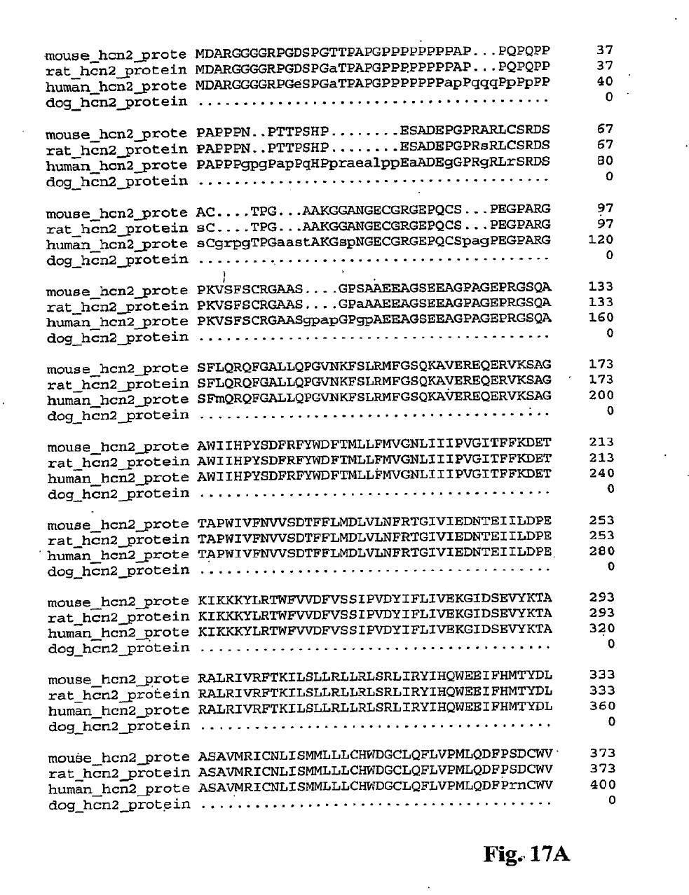

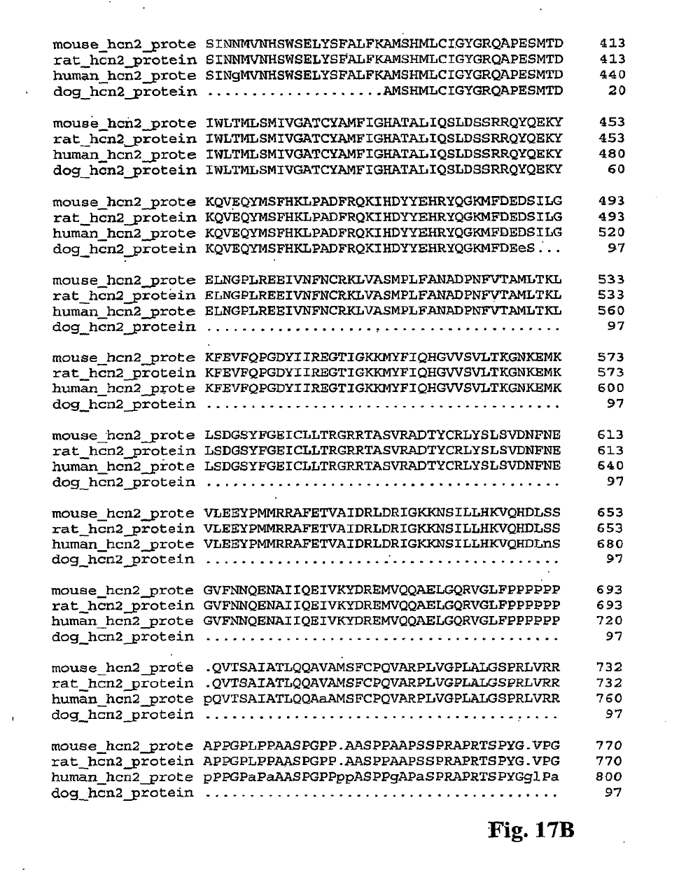

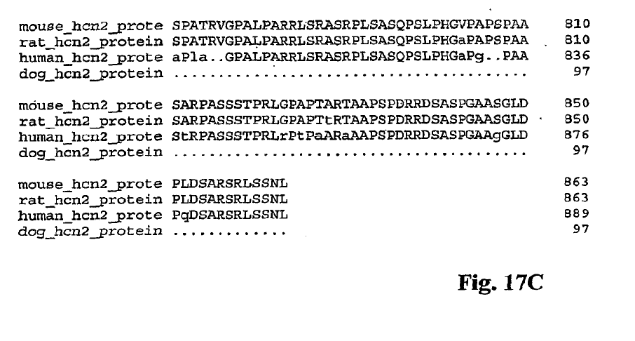

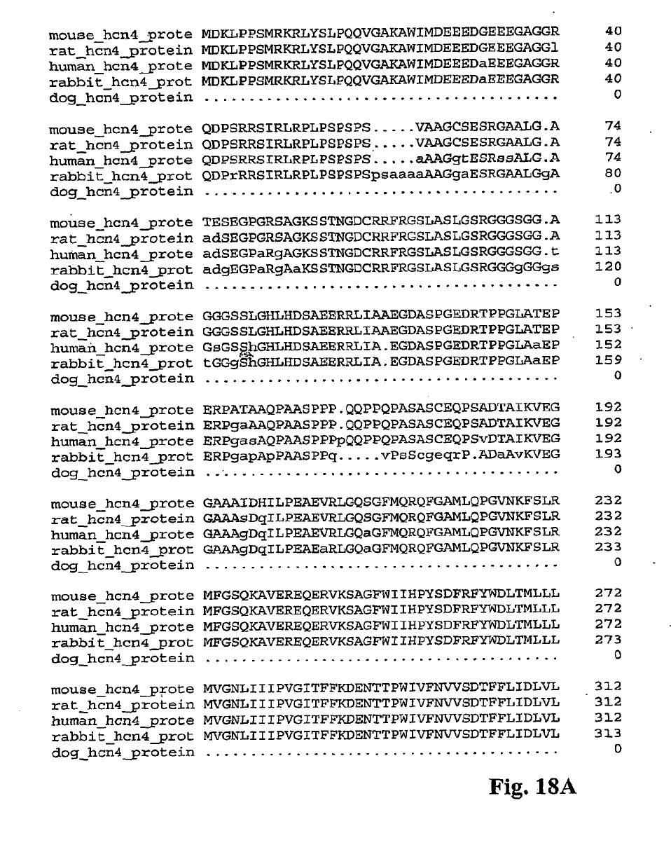

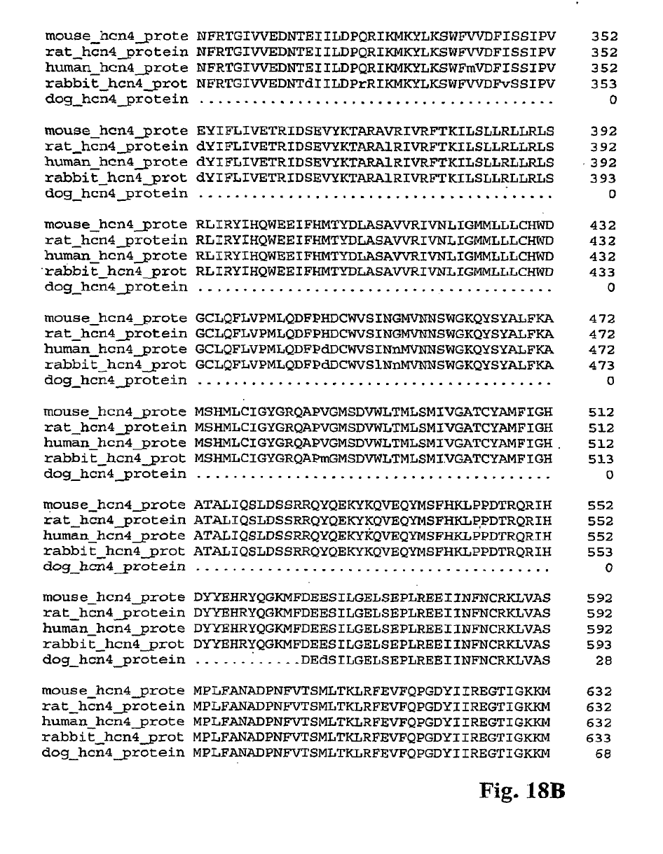

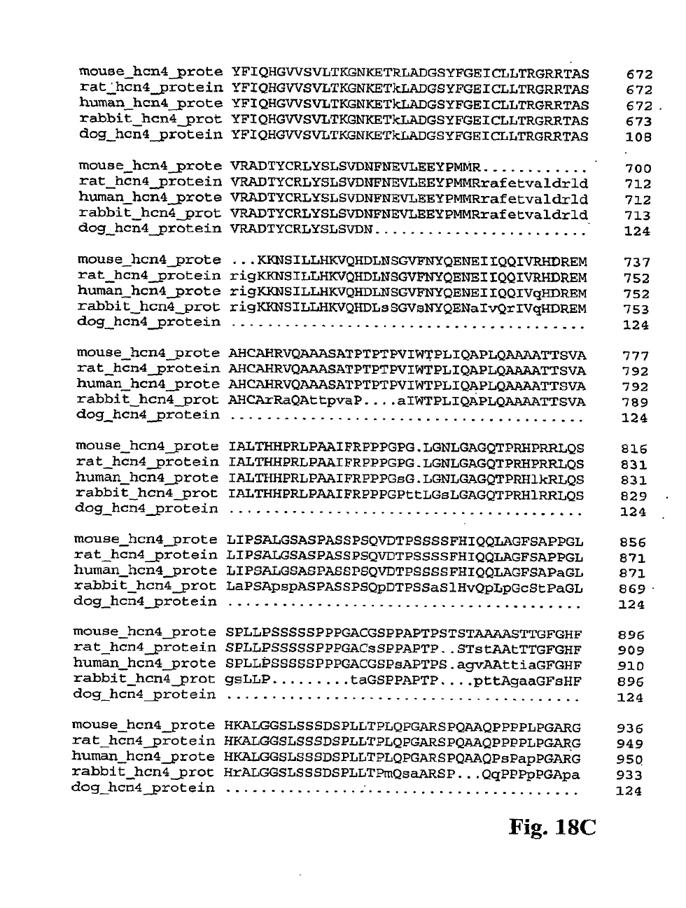

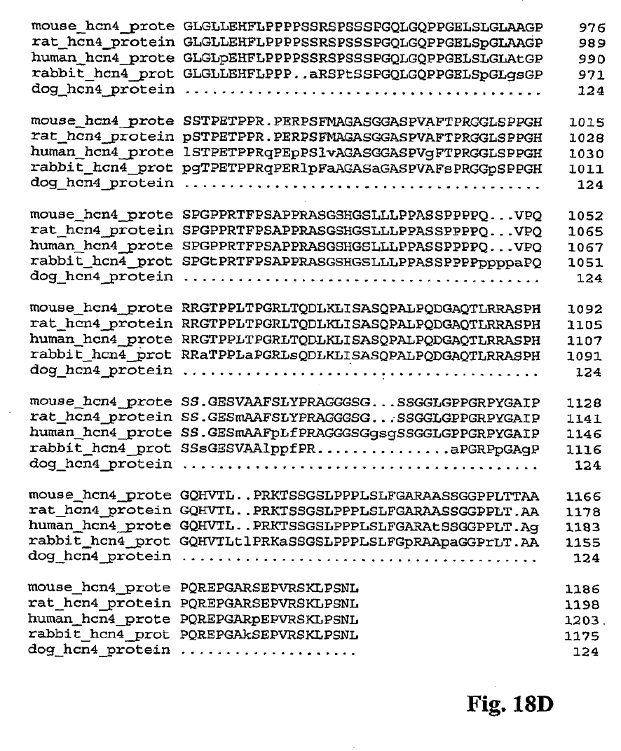

FIG. 13 demonstrates that the biophysical properties of passage 5 and passage 9 cells expressing HCN2-induced current are very similar. FIG. 13A is a comparison of current records of HCN2-incuded current in passage 5 (left panel) and passage 9 (right panel) hMSCs. The current records are very similar. FIG. 13B depicts activation curves obtained from passage 5 (left panel) and passage 9 (right panel) cells show the same midpoint of activation.

What is claimed is:

1. An isolated human adult mesenchymal stem cell, substantially incapable of differentiation, comprising an exogenous molecule.

2. The human adult mesenchymal stem cell of claim 1, wherein the cell is genetically engineered to express a polypeptide or an oligonucleotide.

3. The human adult mesenchymal stem cell of claim 1, wherein the exogenous molecule comprises an oligonucleotide, polypeptide, or small molecule, and wherein said cell is capable of delivering said exogenous molecule to an adjacent cell.

4. The stem cell of claim 2 wherein the oligonucleotide is a siRNA

5. The human adult mesenchymal stem cell of claim 1 wherein said cell has been passaged at least nine times.

6. The mesenchymal stem cell of claim 1, which
(i) has been passaged at least nine times;
(ii) expresses CD29, CD44, CD54 and HLA class I surface markers; and
(iii) do not express CD14, CD45, CD34 and HLA class II surface markers.

7. The mesenchymal stem cell of claim 2, wherein the expression of the polypeptide or oligonucleotide stimulates cardiomyocyte proliferation.

8. The mesenchymal stem cell of claim 2, wherein expression of the polypeptide or oligonucleotide promotes cardiac repair.

9. The mesenchymal stem cell of claim 3 wherein delivery of the exogenous molecule to adjacent cells stimulates cardiomyocyte proliferation.

10. The mesenchymal stem cell of claim 3 wherein delivery of the exogenous molecule to adjacent cells stimulates cardiac repair.

11. The human adult mesenchymal stem cell of claim 3, wherein the cell is capable of gap junction mediated communication with cardiomyocytes.

12. A pharmaceutical composition comprising a population of human adult mesenchymal stem cells, substantially incapable of differentiation, and a pharmaceutically acceptable carrier.

13. The pharmaceutical composition of claim 12, wherein the human adult mesenchymal stem cell cells have been passaged at least nine times.

14. The pharmaceutical composition of claim 12, wherein the population of human adult mesenchymal stem cells:
(i) have been passaged at least nine times;
(ii) express CD29, CD44, CD54 and HLA class I surface markers; and
(iii) do not express CD14, CD45, CD34 and HLA class II surface markers.

15. A pharmaceutical composition comprising a human adult mesenchymal stem cell, substantially incapable of differentiation and comprising an exogenous molecule.

16. The pharmaceutical composition of claim 15 wherein the human adult mesenchymal stem cell is genetically engineered to express an oligonucleotide or a polypeptide.

17. The pharmaceutical composition of claim 16 wherein the oligonucleotide is a siRNA.

18. The pharmaceutical composition of claim 15, wherein the exogenous molecule is an oligonucleotide, polypeptide, or small molecule, and wherein said cell is capable of delivering said exogenous molecule to an adjacent cell.

19. A method for promoting cardiac repair in a subject, comprising administering to said subject an effective amount of human adult mesenchymal stem cells, that are substantially incapable of differentiation, thereby promoting cardiac repair.

20. The method of claim 19 wherein said human adult mesenchymal stem cells have been passaged at least nine times.

21. The method of claim 19, wherein said human adult mesenchymal stem cells are passaged at least nine times and express CD29, CD44, CD54 and HLA class I surface markers and do not express CD14, CD45, CD34 and HLA class II surface markers.

22. The method of claim 19, wherein said human adult mesenchymal stem cells comprise an exogenous molecule.

23. The method of claim 22, wherein said human adult mesenchymal stem cells are genetically engineered to express an oligonucleotide or a polypeptide.

24. The method of claim 23, wherein the oligonucleotide is a siRNA.

25. The method of claim 22, wherein the exogenous molecule comprises an oligonucleotide, polypeptide, or small molecule, and wherein said cell is capable of delivering said exogenous molecule to an adjacent cell.

26. The method of claim 24, wherein said human adult mesenchymal stem cells are genetically engineered to express the Wnt-5A ligand.

27. The method of claim 19, wherein said subject is afflicted with myocardial dysfunction or infarction.

28. A method of delivering an oligonucleotide, protein or small molecule into a target cell comprising:
(i) introducing the oligonucleotide, protein, or small molecule into a human adult mesenchymal stem cell that is substantially incapable of differentiation, and
(ii) contacting the target cell with the stem cell under conditions permitting the stem cell to form a gap junction channel with the target cell, whereby the oligonucleotide, protein of small molecule is delivered into the target cell from the stem cell.
